(19) United States
(12) Patent Application Publication
(10) Pub. No.: US 2024/0033079 A1
(43) Pub. Date: Feb. 1, 2024

(54) IMPLANTABLE MEDICAL DEVICES

(71) Applicant: EDWARDS LIFESCIENCES CORPORATION, Irvine, CA (US)

(72) Inventors: Shahram Zamani, Newport Coast, CA (US); Waina Michelle Chu, Tustin, CA (US); Ziv Yohanan, Kfar Hahoresh (IL); Dikla Kersh, Karkur (IL)

(21) Appl. No.: 18/482,636

(22) Filed: Oct. 6, 2023

Related U.S. Application Data
(63) Continuation of application No. PCT/US2022/023621, filed on Apr. 6, 2022.
(60) Provisional application No. 63/171,746, filed on Apr. 7, 2021.

(51) Int. Cl. A61F 2/24

(52) U.S. Cl. CPC A61F 2/2418; A61F 2250/0018; A61F 2250/0037; A61F 2250/0036; A61F 2240/001

(57) ABSTRACT

Disclosed herein are implantable medical devices comprising a textile material comprising a first material and a second material having different stretchability and flexibility, where the first and the second materials are at least partially superposed and coupled to each other. The medical devices of the current disclosure can be implantable prosthetic valves. The valves of the present disclosure provide for a reduced crimped profile and improve PVL sealing. In addition, disclosed herein are methods of making the implantable medical devices.

### CROSS-REFERENCE TO RELATED APPLICATIONS

This application is a continuation of PCT patent application number PCT/US2022/023621, filed on Apr. 6, 2022, which application claims the benefit of U.S. Provisional Application No. 63/171,746 filed Apr. 7, 2021, the content of each of which is incorporated herein by reference in its entirety.

### FIELD

The present disclosure relates to implantable expandable prosthetic devices and to methods and apparatuses for such prosthetic devices.

### BACKGROUND

The heart can suffer from various valvular diseases or malformations that result in significant malfunctioning of the heart and ultimately require replacing the native heart valve with an artificial one. Human heart valves, which include the aortic, pulmonary, mitral, and tricuspid valves, function essentially as one-way valves operating in synchronization with the pumping heart. The valves allow blood to flow downstream but block blood from flowing upstream. Diseased heart valves exhibit impairments such as narrowing of the valve or regurgitation, which inhibits the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open-heart surgery. Replacement valves are being designed to be delivered through minimally invasive procedures and even percutaneous procedures. Such replacement valves often include a tissue-based valve body that is connected to an expandable frame that is then delivered to the native valve's annulus.

Because of the drawbacks associated with conventional open-heart surgery, percutaneous and minimally invasive surgical approaches are garnering intense attention. In one technique, a prosthetic valve is configured to be implanted in a much less invasive procedure by way of catheterization. For instance, U.S. Pat. Nos. 5,411,522 and 6,730,118, 7,393,360, 7,510,575, and 7,993,394, which are incorporated herein by reference, describe collapsible transcatheter heart valves (THVs) that can be percutaneously introduced in a compressed state on a catheter and expanded in the desired position by balloon inflation or by utilization of a self-expanding frame or stent. In yet another example, U.S. U.S. Publication Nos. 2014/0277390, 2014/0277422, 2014/0277427, and 2015/0328000, and 2019/0328515, which are incorporated herein by reference in their entirety, describe heart valve prostheses for replacing a native mitral valve, including a self-expanding frame with a plurality of anchoring members that are designed be deployed within a body cavity and prevent axial flow of fluid around an exterior of the prosthesis.

An important design parameter of a transcatheter heart valve is the diameter of the folded or crimped profile. The diameter of the crimped profile is important because it directly influences the physician's ability to advance the transcatheter heart valve through the femoral artery or vein. More specifically, the smaller profile allows for the treatment of a wider population of patients with enhanced safety. However, the reduction in the crimped profile can introduce additional challenges. For example, a reduction in the crimped profile can affect the ability of the valve to properly seal with the surrounding cardiac tissue and thus to prevent paravalvular leakage.

An additional challenge relates to the ability of such prostheses to be secured relative to intra-luminal tissue, e.g., tissue within any body lumen or cavity, in an atraumatic manner.

These needs and others are at least partially satisfied by the present disclosure.

### SUMMARY

Some aspects of the present disclosure relate to implantable medical devices. In some aspects, disclosed herein is an implantable prosthetic device comprising: an annular frame having an inflow end, an outflow end, and a longitudinal axis and comprising a plurality of struts; and a textile defined by a first surface and an opposite second surface, having a longitudinal axis and a transverse axis, wherein the longitudinal axis of the textile is substantially parallel to the longitudinal axis of the annular frame; and wherein the textile has a proximal end and a distal end and is mounted circumferentially around a first portion of the annular frame, wherein the first portion has a proximal end and a distal end, wherein the proximal end of the first portion is at the inflow end of the annular frame; wherein at least a portion of the textile comprises at least one composite material comprising a first material and a second material coupled together such that a crimped profile of the implantable medical device is reduced when compared to a substantially identical reference implantable medical device in the absence of the at least one composite material; wherein the first material exhibits a flexibility and stretchability lower than a flexibility and stretchability of the second material; wherein the first material has higher a mechanical strength than a mechanical strength of the second material; and wherein the implantable medical device is configured to radially expand to an expanded configuration from the crimped profile.

Also disclosed are aspects wherein the first material and the second material are at least partially superposed. In some aspects, the first material can be a woven material. While in other aspects, the second material can be a knitted material.

In still further aspects, the first material and the second material can be coupled by an ultrasonic welding such that at least one joining region is formed.

Also disclosed herein are methods of forming an implantable medical device comprising: a) providing an annular frame having an inflow end, an outflow end, and a longitudinal axis and comprising a plurality of struts; b) circumferentially mounting a textile defined by a first surface and an opposite second surface, having a longitudinal axis and a transverse axis, wherein the longitudinal axis of the textile is substantially parallel to the longitudinal axis of the annular frame; and wherein the textile has a proximal end and a distal end and is mounted circumferentially around a first portion of the annular frame, wherein the first portion has a proximal end and a distal end, wherein the proximal end of the first portion is at the inflow end of the annular frame; wherein at least a portion of the textile comprises at least one composite material comprising a first material and a second material coupled together such that a crimp profile of the implantable medical device is reduced when compared to a substantially identical reference implantable medical device in the absence of the at least one composite material; wherein the first material exhibits a flexibility and stretchability lower than a flexibility and stretchability of the second material; wherein the first material has higher a mechanical strength than a mechanical strength of the second material, and wherein the implantable medical device is configured to radially expand to an expanded configuration from the crimp profile.

Additional aspects of the disclosure will be set forth, in part, in the detailed description, figures, and claims which follow, and in part will be derived from the detailed description or can be learned by practice of the invention. It is to be understood that both the foregoing general description and the following detailed description are exemplary and explanatory only and are not restrictive of the invention as disclosed.

### BRIEF DESCRIPTION OF THE DRAWINGS

FIG. 1 is a perspective view of an exemplary valve in one aspect.

FIG. 2 is a perspective view of an exemplary textile in one aspect.

FIG. 3 is a perspective view of an exemplary textile in one aspect

FIG. 4 is a perspective view of an exemplary textile in one aspect.

FIGS. 5-11 show an exemplary frame of the prosthetic heart valve.

FIG. 12 shows an exemplary inner skirt in one aspect.

FIG. 13 shows an exemplary prosthetic heart valve in one aspect.

FIGS. 14A-14B show an exemplary outer skirt that can be used in the exemplary prosthetic heart valve of FIG. 13.

FIG. 15 shows an exemplary attachment at the outer skirt to the frame.

FIG. 16 shows the exemplary prosthetic heart valve of FIG. 13 in the crimped configuration.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

As used in this application and in the claims, the singular forms “a,” “an,” and “the” include the plural forms unless the context clearly dictates otherwise. Thus, for example, reference to a “fiber” includes aspects having two or more such fibers unless the context clearly indicates otherwise.

The term “comprising” and variations thereof as used herein is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples of the invention and are also disclosed. Other than in the examples, or where otherwise noted, all numbers expressing quantities of ingredients, reaction conditions, and so forth used in the specification and claims are to be understood at the very least and not as an attempt to limit the application of the doctrine of equivalents to the scope of the claims, to be construed in light of the number of significant digits and ordinary rounding approaches.

For the terms “for example,” “exemplary,” and “such as,” and grammatical equivalences thereof, the phrase “and without limitation” is understood to follow unless explicitly stated otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint, and independently of the other endpoint.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

As used herein, the terms “optional” or “optionally” mean that the subsequently described event or circumstance may or may not occur and that the description includes instances where said event or circumstance occurs and instances where it does not.

Further, the terms “coupled” and “associated” generally mean electrically, electromagnetically, and/or physically (e.g., mechanically or chemically) coupled or linked and do not exclude the presence of intermediate elements between the coupled or associated items.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation. Although the operations of exemplary aspects of the disclosed method may be described in a particular sequential order for convenient presentation, it should be understood that disclosed aspects can encompass an order of operations other than the particular sequential order disclosed. For example, operations described sequentially may, in some cases, be rearranged or performed concurrently. Further, descriptions and disclosures provided in association with one particular aspect are not limited to that aspect and may be applied to any aspect disclosed.

The term “fiber” as used herein includes fibers of extreme or indefinite length (i.e., filaments) and fibers of short length (i.e., staple fibers).

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”). As used herein, the term “and/or” includes any and all combinations of one or more of the associated listed items.

It will be understood that, although the terms “first,” “second,” etc., may be used herein to describe various elements, components, regions, layers and/or sections, and/or steps. These elements, components, regions, layers, and/or sections and/or steps should not be limited by these terms. These terms are only used to distinguish one element, component, region, layer, or section from another element, component, region, layer, section, or step. Thus, a first element, component, region, layer, section, or step discussed below could be termed a second element, component, region, layer, section, or step without departing from the teachings of example aspects.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

As used herein, the term “substantially” means that the subsequently described event or circumstance completely occurs or that the subsequently described event or circumstance generally, typically, or approximately occurs.

Still further, the term “substantially” can in some aspects refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

In other aspects, as used herein, the term “substantially free,” when used in the context of a composition or component of a composition that is substantially absent, is intended to indicate that the recited component is not intentionally batched and added to the composition, but can be present as an impurity along with other components being added to the composition. In such aspects, the term “substantially free,” is intended to refer to trace amounts that can be present in the batched components, for example, it can be present in an amount that is less than about 1% by weight, e.g., less than about 0.5% by weight, less than about 0.1% by weight, less than about 0.05% by weight, or less than about 0.01% by weight of the stated material, based on the total weight of the composition.

As used herein, the term “substantially,” in, for example, the context “substantially identical” or “substantially similar” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

As used herein, the terms “substantially identical reference composition” or “substantially identical reference article” refer to a reference composition or article comprising substantially identical components in the absence of an inventive component. In another exemplary aspect, the term “substantially,” in, for example, the context “substantially identical reference composition,” refers to a reference composition comprising substantially identical components and wherein an inventive component is substituted with a common in the art component. For example, a substantially identical reference sheath can comprise a sheath comprising substantially identical components but without the presence of the at least one composite material as described.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

### Implantable Medical Device

The aspects of the current disclosure refer to implantable medical devices. Any known in the art medical devices can have the disclosed herein features. However, in some aspects, the implantable prosthetic devices disclosed herein refer to a heart valve.

In certain aspects, disclosed herein is an implantable medical device comprising: an annular frame having an inflow end, an outflow end, and a longitudinal axis and comprising a plurality of struts; and a textile defined by a first surface and an opposite second surface, having a longitudinal axis and a transverse axis, wherein the longitudinal axis of the textile is substantially parallel to the longitudinal axis of the annular frame; and wherein the textile has a proximal end and a distal end and is mounted circumferentially around a first portion of the annular frame, wherein the first portion has a proximal end and a distal end, wherein the proximal end of the first portion is at the inflow end of the annular frame; wherein at least a portion of the textile comprises at least one composite material comprising a first material and a second material coupled together such that a crimped profile of the implantable medical device is reduced when compared to a substantially identical reference implantable medical device in the absence of the at least one composite material; wherein the first material exhibits a flexibility and stretchability lower than a flexibility and stretchability of the second material; wherein the first material has higher a mechanical strength than a mechanical strength of the second material; and wherein the implantable medical device is configured to radially expand to an expanded configuration from the crimped profile.

In still further aspects, the implantable medical device is an implantable prosthetic valve. In yet still further aspects, the implantable prosthetic valve is any of the heart valves disclosed below.

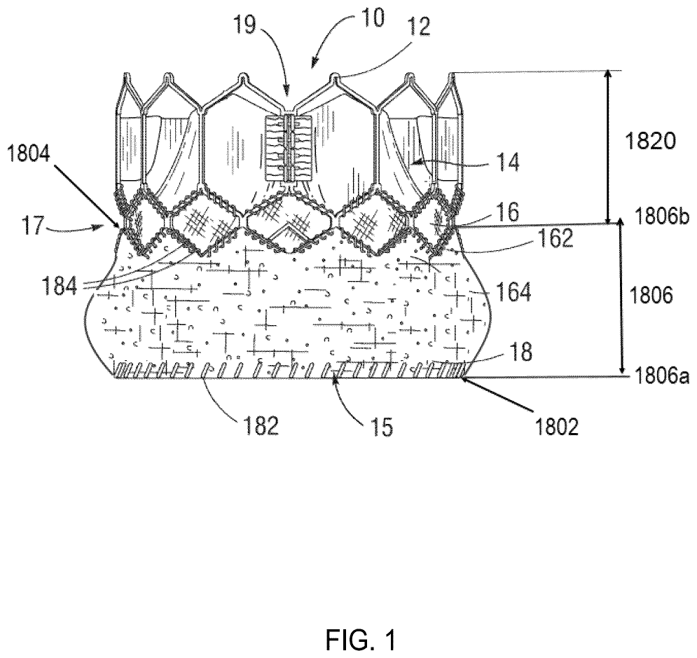

FIG. 1 shows an exemplary prosthetic heart valve 10, according to one aspect. The illustrated prosthetic valve is adapted to be implanted in the native aortic annulus, although in other aspects, it can be adapted to be implanted in the other native annuluses of the heart (e.g., the pulmonary, mitral, and tricuspid valves). The prosthetic valve can also be adapted to be implanted in other tubular organs or passageways in the body. The prosthetic valve 10 can have four main components: an annular stent or frame 12, a valvular structure 14, an inner skirt 16, and an exemplary perivalvular outer sealing member or outer skirt 18. The exemplary prosthetic valve 10 has an inflow end portion 15, an intermediate portion 17, and an outflow end portion 19.

In some aspects and as disclosed in detail below, the textile can be used as an inner skirt, as an outer skirt, or both.

In some aspects, the inner skirt 16 is mounted on an inner surface of the annular frame 12. Yet, in other aspects, the outer skirt 18 can be mounted on an outer surface of the annular frame 12.

In still further aspects, the outer sealing member 18 has a proximal end 1802 and a distal end 1804 and is mounted circumferentially around a first portion of the outer surface of the annular frame 12, wherein the first portion 1806 of the outer surface has a proximal end and a distal end, wherein the proximal end of the first portion is at the inflow end 15 of the annular frame 12. In aspects disclosed herein, for example, the first portion 1806 of the annular frame can be between the inflow end 15 of the annular frame and the beginning of the intermediate portion 17 of the frame. The first portion, 1806, is also defined by a proximal end 1806a and a distal end 1806b.

In still further aspect, the annular frame also has a second portion 1820 that is free of the outer sealing member and extends between the outflow end 19 of the annular frame and the distal end of the first portion 1806b.

The valvular structure 14 can comprise three leaflets collectively forming a leaflet structure, which can be arranged to collapse in a tricuspid arrangement. The lower edge of leaflet structure 14 desirably has an undulating, curved scalloped shape (not shown). By forming the leaflets with this scalloped geometry, stresses on the leaflets are reduced, which in turn improves the durability of the prosthetic valve. Moreover, by virtue of the scalloped shape, folds and ripples at the belly of each leaflet (the central region of each leaflet), which can cause early calcification in those areas, can be eliminated or at least minimized. The scalloped geometry also reduces the amount of tissue material used to form leaflet structure, thereby allowing a smaller, more even crimped profile at the inflow end of the prosthetic valve. The leaflets 14 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

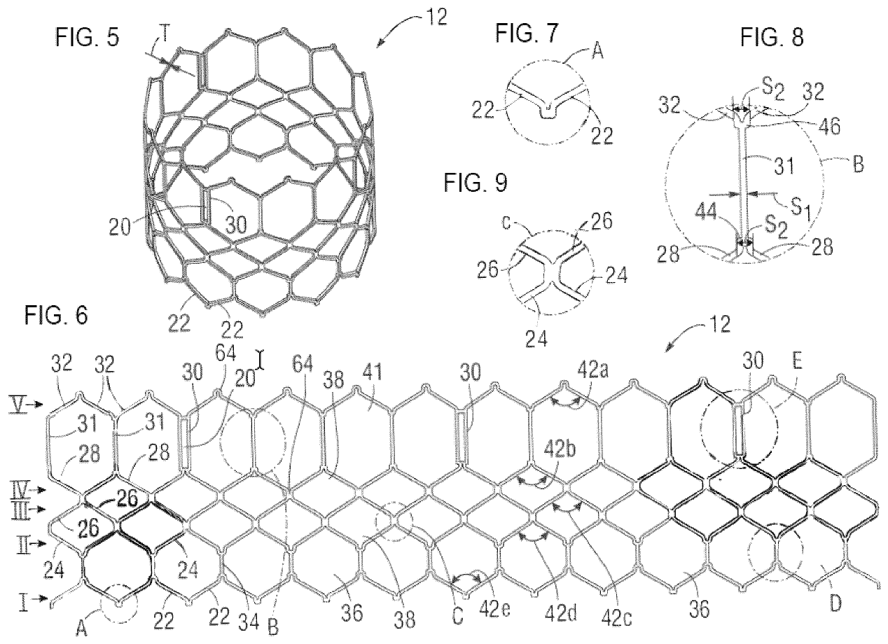

Any known in the art of medical implantable devices annular frames can be used. The bare, exemplary and unlimiting frame 12 is shown in FIG. 5. Frame 12 can be formed with a plurality of circumferentially spaced slots or commissure windows, 20 (three in the illustrated and unlimiting aspect) that are adapted to mount the commissures of the valvular structure 14 to the frame. The frame 12 can be made of any of various suitable plastically-expandable materials (e.g., stainless steel, etc.) or self-expanding materials (e.g., nickel titanium alloy (NiTi), such as nitinol) as known in the art. When constructed of a plastically-expandable material, frame 12 (and thus the prosthetic valve 10) can be crimped to a radially collapsed configuration on a delivery catheter and then expanded inside a patient by an inflatable balloon or equivalent expansion mechanism. When constructed of a self-expandable material, frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include, without limitation, stainless steel, biocompatible, high-strength alloys (e.g., a cobalt-chromium or a nickel-cobalt-chromium alloys), polymers, or combinations thereof. In particular aspects, frame 12 is made of a nickel-cobalt-chromium-molybdenum alloy, such as MP35N® alloy (SPS Technologies, Jenkintown, Pa.), which is equivalent to UNS R30035 alloy (covered by ASTM F562-02). MP35N® alloy/UNS R30035 alloy comprises 35% nickel, 35% cobalt, 20% chromium, and 10% molybdenum by weight. It has been found that the use of MP35N® alloy to form frame 12 provides superior structural results over stainless steel. In particular, when MP35N® alloy is used as the frame material, less material is needed to achieve the same or better performance in radial and crush force resistance, fatigue resistance, and corrosion resistance. Moreover, since less material is required, the crimped profile of the frame can be reduced, thereby providing a lower profile prosthetic valve assembly for percutaneous delivery to the treatment location in the body.

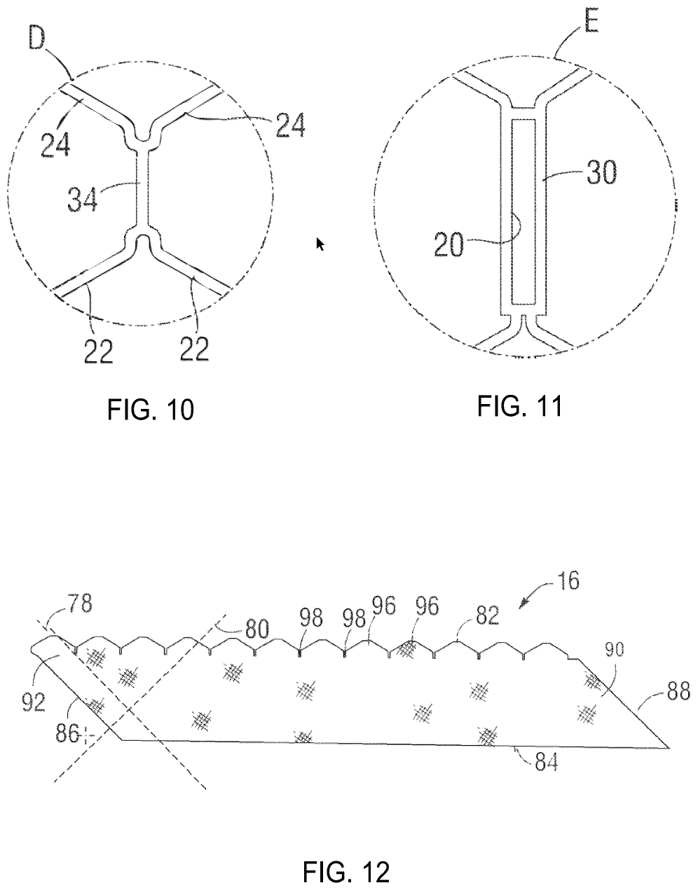

In still further aspects and as disclosed herein, the annular frame further comprises a plurality of struts. Referring to FIGS. 5 and 6, an exemplary frame 12, can comprise a first, lower row I of angled struts 22 arranged end-to-end and extending circumferentially at the inflow end of the frame; a second row II of circumferentially extending angled struts 24; a third row III of circumferentially extending, angled struts 26; a fourth row IV of circumferentially extending angled struts 28; and a fifth row V of circumferentially extending, angled struts 32 at the outflow end of the frame. A plurality of substantially straight axially extending struts 34 can be used to interconnect the struts 22 of the first row I with the struts 24 of the second row II. The fifth row V of angled struts 32 are connected to the fourth row IV of angled struts 28 by a plurality of axially extending window frame portions 30 (which define the commissure windows 20) and a plurality of axially extending struts 31. Each axial strut 31 and each frame portion 30 extends from a location defined by the convergence of the lower ends of two angled struts 32 to another location defined by the convergence of the upper ends of two angled struts 28. FIGS. 7, 8, 9, 10, and 11 are enlarged views of the portions of frame 12 identified by letters A, B, C, D, and E, respectively, in FIG. 6.

Each commissure window frame portion 30 mounts a respective commissure of the leaflet structure 14. As can be seen, each frame portion 30 is secured at its upper and lower ends to the adjacent rows of struts to provide a robust configuration that enhances fatigue resistance under cyclic loading of the prosthetic valve compared to known, cantilevered struts for supporting the commissures of the leaflet structure. This configuration enables a reduction in the frame wall thickness to achieve a smaller crimped diameter of the prosthetic valve. In particular aspects, the thickness T of frame 12 (FIG. 5) measured between the inner diameter and outer diameter is about 0.48 mm or less.

The struts and frame portions of the frame collectively define a plurality of open cells of the frame. At the inflow end of frame 12, struts 22, struts 24, and struts 34 define a lower row of cells defining openings 36. The second, third, and fourth rows of struts 24, 26, and 28 define two intermediate rows of cells defining openings 38. The fourth and fifth rows of struts 28 and 32, along with frame portions 30 and struts 31, define an upper row of cells defining openings 40. The openings 41 are relatively large and are sized to allow portions of the leaflet structure 14 to protrude, or bulge, into and/or through the openings 40 when the frame 12 is crimped in order to minimize the crimping profile.

As best shown in FIG. 8, the lower end of the strut 31 is connected to two struts 28 at a node or junction 44, and the upper end of the strut 31 is connected to two struts 32 at a node or junction 46. The strut 31 can have a thickness S₁ that is less than the thicknesses S₂ of the junctions 44, 46. The junctions 44, 46, along with junctions 64, prevent full closure of openings 40. The geometry of the struts 31, and junctions 44, 46, and 64 assists in creating enough space in openings 41 in the collapsed configuration to allow portions of the prosthetic leaflets to protrude or bulge outwardly through openings. This allows the prosthetic valve to be crimped to a relatively smaller diameter than if all of the leaflet material were constrained within the crimped frame.

Frame 12 is configured to reduce, prevent, or minimize possible over-expansion of the prosthetic valve at a predetermined balloon pressure, especially at the outflow end portion of the frame, which supports the leaflet structure 14. In one aspect, the frame is configured to have relatively larger angles 42a, 42b, 42c, 42d, 42e between struts, as shown in FIG. 6. The larger the angle, the greater the force required to open (expand) the frame. As such, the angles between the struts of the frame can be selected to limit the radial expansion of the frame at a given opening pressure (e.g., inflation pressure of the balloon). In particular aspects, these angles are at least 110 degrees or greater when the frame is expanded to its functional size, and even more particularly, these angles are up to about 120 degrees when the frame is expanded to its functional size.

In addition, the inflow 15 and outflow 19 ends of the annular frame generally tend to over-expand more so than the middle portion of the frame due to the “dog-boning” effect of the balloon used to expand the prosthetic valve. To protect against over-expansion of the leaflet structure 14, the leaflet structure desirably is secured to the frame 12 below the upper row of struts 32, as best shown in FIG. 1. Thus, in the event that the outflow end of the frame is over-expanded, the leaflet structure is positioned at a level below where over-expansion is likely to occur, thereby protecting the leaflet structure from over-expansion.

In a known prosthetic valve construction, portions of the leaflets can protrude longitudinally beyond the outflow end of the frame when the prosthetic valve is crimped if the leaflets are mounted too close to the distal end of the frame. If the delivery catheter on which the crimped prosthetic valve is mounted includes a pushing mechanism or stop member that pushes against or abuts the outflow end of the prosthetic valve (for example, to maintain the position of the crimped prosthetic valve on the delivery catheter), the pushing member or stop member can damage the portions of the exposed leaflets that extend beyond the outflow end of the frame. Another benefit of mounting the leaflets at a location spaced away from the outflow end of the frame is that when the prosthetic valve is crimped on a delivery catheter, the outflow end of the frame 12 rather than the leaflets is the proximal-most component of the prosthetic valve 10. As such, if the delivery catheter includes a pushing mechanism or stop member that pushes against or abuts the outflow end of the prosthetic valve, the pushing mechanism or stop member contacts the outflow end of the frame, and not leaflets, so as to avoid damage to the leaflets.

Also, as can be seen in FIG. 6, the openings 36 of the lowermost row of openings in the frame are relatively larger than the openings 38 of the two intermediate rows of openings. This allows the frame, when crimped, to assume an overall tapered shape that tapers from a maximum diameter at the outflow end of the prosthetic valve to a minimum diameter at the inflow end of the prosthetic valve. When crimped, frame 12 has a reduced diameter region extending along a portion of the frame adjacent the inflow end of the frame that generally corresponds to the region of the frame covered by the outer skirt 18. Again, it is understood that the frame 12 shown herein is exemplary and unlimiting.

The main functions of the inner skirt 16 are to assist in securing the valvular structure 14 to the frame 12 and to assist in forming a good seal between the prosthetic valve and the native annulus by blocking the flow of blood through the open cells of the frame 12 below the lower edge of the leaflets. The inner skirt 16 desirably comprises a tough, tear-resistant material such as polyethylene terephthalate (PET), although various other synthetic materials or natural materials (e.g., pericardial tissue) can be used. The thickness of the skirt desirably is less than about 0.15 mm (about 6 mil), and desirably less than about 0.1 mm (about 4 mil), and even more desirably about 0.05 mm (about 2 mil). In particular aspects, inner skirt 16 can have a variable thickness, for example, the skirt can be thicker at at least one of its edges than at its center. In one implementation, inner skirt 16 can comprise a PET skirt having a thickness of about 0.07 mm at its edges and about 0.06 mm at its center. The thinner skirt can provide for better crimping performances while still providing good perivalvular sealing.

In some aspects, the reduced diameter region is reduced compared to the diameter of the upper portion of the frame (which is not covered by the outer skirt) such that the outer skirt 18 does not increase the overall crimped profile of the prosthetic valve. When the prosthetic valve is deployed, the frame can expand to the generally cylindrical shape shown in FIG. 5. In one example, the frame of a 26-mm prosthetic valve, when crimped, had a first diameter of 14 French at the outflow end of the prosthetic valve and a second diameter of 12 French at the inflow end of the prosthetic valve.

The inner skirt 16 can be secured to the inside of frame 12 via sutures. Valvular structure 14 can be attached to the skirt via one or more reinforcing strips (which collectively can form a sleeve), for example, thin, PET reinforcing strips, discussed below, which enables a secure suturing and protects the pericardial tissue of the leaflet structure from tears. Valvular structure 14 can be sandwiched between skirt 16 and these reinforcing strips. Additional sutures can be used to secure the PET strip and the leaflet structure 14 to skirt 16, can be any suitable suture, such as Ethibond Excel® PET suture (Johnson & Johnson, New Brunswick, N.J.).

Known fabric inner skirts can comprise a weave of warp and weft fibers that extend perpendicularly to each other and with one set of fibers extending longitudinally between the upper and lower edges of the skirt. When the metal frame to which the fabric of the inner skirt is secured is radially compressed, the overall axial length of the frame increases. Unfortunately, an inner skirt with limited elasticity cannot elongate along with the frame and therefore tends to deform the struts of the frame and to prevent uniform crimping.

Any known in the art fabrics that are suitable for the disclosed herein purposes can be used as an inner skirt. An exemplary and unlimiting inner skirt 16 is shown in FIG. 12. The exemplary inner skirt 16 as disclosed herein can be woven from a first set of fibers, or yarns or strands, 78 and a second set of fibers, or yarns or strands, 80, both of which are non-perpendicular to the upper edge 82 and the lower edge 84 of the skirt. In particular aspects, the first set of fibers 78 and the second set of fibers 80 extend at angles of about 45 degrees relative to the upper and lower edges 82, 84. Alternatively, the first set of fibers 78 and the second set of fibers 80 extend at angles other than about 45 degrees relative to the upper and lower edges 82, 84, e.g., at angles of 15 and 75 degrees, respectively, or 30 and 60 degrees, respectively, relative to the upper and lower edges 82, 84. For example, the inner skirt 16 can be formed by weaving the fibers at 45 degree angles relative to the upper and lower edges of the fabric. Alternatively, the inner skirt 16 can be diagonally cut (cut on a bias) from a vertically woven fabric (where the fibers extend perpendicularly to the edges of the material) such that the fibers extend at 45 degree angles relative to the cut upper and lower edges of the skirt. As further shown in FIG. 12, the opposing short edges 86, 88 of the inner skirt can be, for example, non-perpendicular to the upper and lower edges 82, 84. In another example, the short edges 86 and/or 88 can extend at angles of about 45 degrees relative to the upper and lower edges and therefore are aligned with the first set of fibers 78. Therefore the overall general shape of the inner skirt is that of a rhomboid or parallelogram. Without wishing to be bound by any theory, it is noted that the 45 degree angle orientation can provide dimensional compliance to a rigid woven cloth as the inner skirt sees dimensional changes during crimping and expansion.

Due to the angled orientation of the fibers relative to the upper and lower edges, the inner skirt can undergo greater elongation in the axial direction (i.e., in a direction from the upper edge 82 to the lower edge 84). Thus, when the metal frame 12 is crimped, the inner skirt 16 can elongate in the axial direction along with the frame and therefore provide a more uniform and predictable crimping profile. Each cell of the metal frame in the illustrated aspect includes at least four angled struts that rotate towards the axial direction on crimping (e.g., the angled struts become more aligned with the length of the frame). The angled struts of each cell function as a mechanism for rotating the fibers of the skirt in the same direction of the struts, allowing the skirt to elongate along the length of the struts. This allows for greater elongation of the skirt and avoids undesirable deformation of the struts when the prosthetic valve is crimped.

In addition, the spacing between the woven fibers or yarns can be increased to facilitate the elongation of the skirt in the axial direction. For example, for a PET inner skirt 16 formed from 20-denier yarn, the yarn density can be about 15% to about 30% lower than in a typical PET skirt. In some examples, the yarn spacing of the inner skirt 16 can be from about 60 yarns per cm (about 155 yarns per inch) to about 70 yarns per cm (about 180 yarns per inch), such as about 63 yarns per cm (about 160 yarns per inch), whereas in a typical PET skirt the yarn spacing can be from about 85 yarns per cm (about 217 yarns per inch) to about 97 yarns per cm (about 247 yarns per inch). The oblique edges 86, 88 promote a uniform and even distribution of the fabric material along an inner circumference of the frame during crimping so as to reduce or minimize bunching of the fabric to facilitate uniform crimping to the smallest possible diameter. Additionally, cutting diagonal sutures in a vertical manner may leave loose fringes along the cut edges. The oblique edges 86 88 help minimize this from occurring. Compared to the construction of a typical skirt (fibers running perpendicularly to the upper and lower edges of the skirt), the construction of the inner skirt 16 avoids undesirable deformation of the frame struts and provides more uniform crimping of the frame.

In alternative aspects, the inner skirt can be formed from woven elastic fibers that can stretch in the axial direction during crimping of the prosthetic valve. The warp and weft fibers can run perpendicularly and parallel to the upper and lower edges of the skirt, or alternatively, they can extend at angles between 0 and 90 degrees relative to the upper and lower edges of the skirt as described above.

In certain aspects, the textile comprising a composite material as disclosed herein can also be used as an inner skirt. In such aspects, the textile can be disposed on an inner surface of the annular frame. In still further aspects, the disclosed herein textile comprising the disclosed herein composite material can be configured to be used simultaneously as an inner skirt and the outer skirt. In such exemplary and unlimiting aspects, the composite material can be configured such that the first material is disposed on the inner surface of the annular frame and the second material is disposed on the outer surface of the annular frame. In such aspects, the annular frame can be sandwiched between the first and second materials. In still further aspects, the first and second material can be coupled to each other by any means as disclosed below. It is understood that in such exemplary aspects, the first and the second materials can be coupled after mounting on the frame. In yet further aspects, the textile comprising the disclosed herein composite material can be disposed on an outer surface of the annular frame and is an outer skirt. In yet still further aspects, the outer skirt is a sealing member configured to prevent a paravalvular leak.

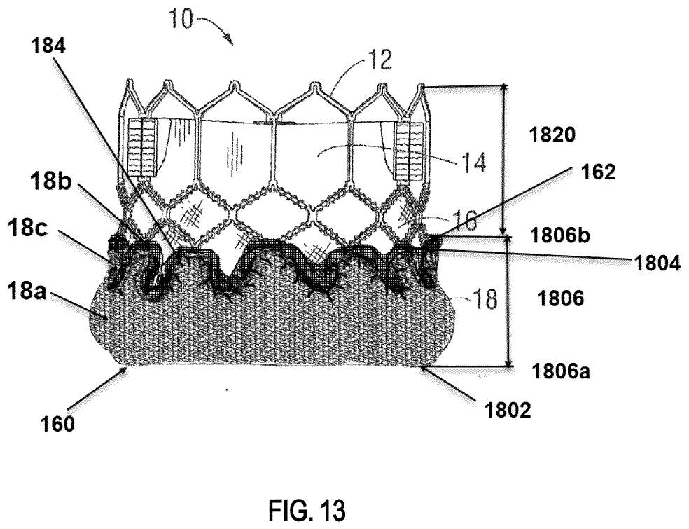

In aspects where the disclosed herein textile is the outer skirt, the proximal end of the textile can be coupled to the proximal end 1806a of the first portion of the annular frame. In yet other aspects, the distal end of the textile can be coupled to the distal end 1806b of the first portion of the annular frame (as, for example, shown in FIG. 13).

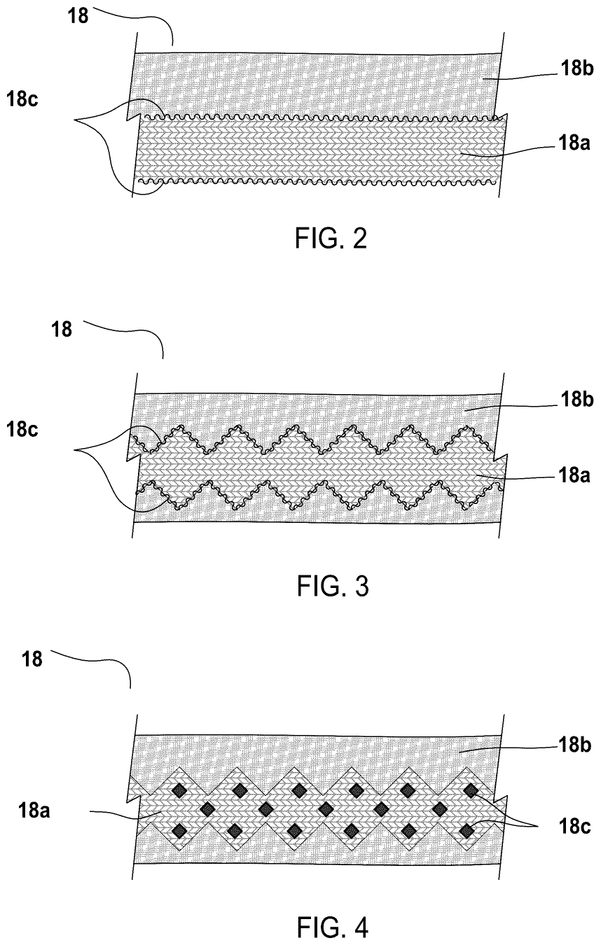

In certain aspects, the first and the second material can be at least partially superposed. Some exemplary configurations of the composite material are shown in FIGS. 2-4.

In certain aspects, the second material is superposed on the first material. It is understood that in yet other aspects (not shown), the first material can be superposed on the second material. It is also understood that the two materials can be superposed on each other in any pattern or configuration. It is understood that the first and second materials can comprise any materials suitable for the discussed purposes and known in the art, as long as a stretchability and flexibility of the first material is lower than those of the second material and/or a mechanical strength of the first material is greater than a mechanical strength of the second material.

In some exemplary and unlimiting aspects, the first material can be a woven material. While in other aspects, the second material can be a knitted material.

Useful woven patterns can include a simple weave, a basket weave, a twill weave, a satin weave, a velour weave, a double velour weave, and combinations thereof. In still further exemplary aspects, the woven material is velour. Useful knitted patterns can include a lock nit pattern, a reverse lock nit pattern, a velour pattern, a double velour pattern, a high-stretch knit pattern having at least a two-needle underlap with a one needle overlap, and combinations thereof. In still further aspects, the knitted material is a crochet knit or a warp-knit fabric.

In some aspects, the woven and/or knitted materials are single-layered. While in other aspects, the woven and/or knitted materials are multi-layered.

In still further aspects, the woven material can comprise a plurality of warp and weft yarns. In such aspects, the warp yarn and/or weft yarn can be fully-drawn, spin drawn, low- or not-twisted, twisted yarn, a flat yarn, a textured yarn, or any combination thereof. In yet other aspects, any combinations of the disclosed herein or other known yarns can be utilized. In certain aspects, the woven material can be the material disclosed above for use in the inner skirt 16.

In still further aspects, the weft and/or warp yarn can have any size suitable for the desired application. For example and without limitations, the warp yarn can have a size from about 5 denier to about 200 denier, including exemplary values of about 7 denier, about 10 denier, about 12 denier, about 15 denier, about 18 denier, about 20 denier, about 22 denier, about 25 denier, about 28 denier, about 30 denier, about 32 denier, about 35 denier, about 38 denier, about 40 denier, about 42 denier, about 45 denier, about 48 denier, about 50 denier, about 52 denier, about 55 denier, about 58 denier, about 60 denier, about 65 denier, about 70 denier, about 75 denier, about 80 denier, about 85 denier, about 90 denier, about 95 denier, about 100 denier, about 120 denier, about 130 denier, about 140 denier, about 150 denier, and about 180 denier. It is understood that the yarn can have any denier values that fall between any two foregoing values.

In still further aspects, the weft and/or warp yarn can have any filament count. For example and without limitations, the weft and/or warp yarn used herein can have a filament count from about 5 to about 200, including exemplary values of about 6, about 7, about 8, about 9, about 10, about 12, about 15, about 18, about 20, about 22, about about 28, about 30, about 32, about 35, about 38, about 40, about 42, about 45, about 48, about 55, about 52, about 55, about 58, about 60, about 62, about 65, about 68, about 70, about 72, about 75, about 78, about 80, about 82, about 85, about 88, about 90, about 92, about 95, and about 98, about 100, about 110, about 120, about 130, about 140, about 150, about 160, about 170, about 180, and about 190. It is understood that the yarn can have any filament count that falls between any two foregoing values. In still further aspects, the weft and/or warp yarn can have a filament count greater than 200, and for example, it can be 220, 250, 280, 300, or greater than 350. Again, it is understood that the specific filament count can be determined based on the desired crimped profile and the PVL seal.

In still further aspects, the weft and/or warp yarn can a size from about 5 denier to about 200 denier, including exemplary values of about 7 denier, about 10 denier, about 12 denier, about 15 denier, about 18 denier, about 20 denier, about 22 denier, about 25 denier, about 28 denier, about 30 denier, about 32 denier, about 35 denier, about 38 denier, about 40 denier, about 42 denier, about 45 denier, about 48 denier, about 50 denier, about 52 denier, about 55 denier, about 58 denier, about 60 denier, about 65 denier, about 70 denier, about 75 denier, about 80 denier, about 85 denier, about 90 denier, about 95 denier, about 100 denier, about 120 denier, about 130 denier, about 140 denier, about 150 denier, and about 180 denier, and a filament count from about 5 to about 200, including exemplary values of about 6, about 7, about 8, about 9, about 10, about 12, about 15, about 18, about 20, about 22, about 25, about 28, about about 32, about 35, about 38, about 40, about 42, about 45, about 48, about 55, about 52, about 55, about 58, about 60, about 62, about 65, about 68, about 70, about 72, about 75, about 78, about 80, about 82, about 85, about 88, about 90, about 92, about 95, and about 98, about 100, about 110, about 120, about 130, about 140, about 150, about 160, about 170, about 180, and about 190.

In some exemplary and unlimiting aspects, the warp and/or weft yarn can be formed by a combination of the twisted yarn and/or flat yarn with the textured yarn, wherein the twisted yarn and/or flat yarn can have a size from about 5 denier to about denier, including exemplary values of about 7 denier, about 10 denier, 12 denier, about 15 denier, about 18 denier, about 20 denier, about 22 denier, about 25 denier, about 28 denier, about 30 denier, about 32 denier, about 35 denier, about 38 denier, about 40 denier, about 45 denier, about 50 denier, about 55 denier, and about 58 denier, and wherein the textured yarn has a size from about 20 denier to about 160 denier, including exemplary values of about 30 denier, about 40 denier, about 50 denier, about 60 denier, about 70 denier, about 80 denier, about 90 denier, about 100 denier, about 120 denier, about 130 denier, about 140 denier, and about 150 denier.

In still further aspects, the woven material can have a tenacity from about 20 cN/tex to about 500 cN/tex, including exemplary values of about 22 cN/tex, about 25 cN/tex, about 28 cN/tex, about 30 cN/tex, about 32 cN/tex, about 35 cN/tex, about 38 cN/tex, about 40 cN/tex, about 42 cN/tex, about 45 cN/tex, about 48 cN/tex, about 50 cN/tex, about 52 cN/tex, about 55 cN/tex, about 58 cN/tex, about 60 cN/tex, about 62 cN/tex, about 65 cN/tex, about 68 cN/tex, about 70 cN/tex, about 72 cN/tex, about 75 cN/tex, about 80 cN/tex, d about 82 cN/tex, about 85 cN/tex, about 90 cN/tex, about 95 cN/tex, about 100 cN/tex, about 110 cN/tex, about 150 cN/tex, about 180 cN/tex, about 200 cN/tex, about 220 cN/tex, about 250 cN/tex, about 280 cN/tex, about 300 cN/tex, about 320 cN/tex, about 350 cN/tex, about 380 cN/tex, about 400 cN/tex, about 420 cN/tex, about 450 cN/tex, and about 480 cN/tex. It is understood that the yarn can have any tenacity value between any two foregoing values.

In still further aspects, the woven material can have permeability from 0 to about 15000 ml/min/cm² of water at a pressure of 120 mm of mercury, including exemplary values of about 5 ml/min/cm², about 10 ml/min/cm², about 20 ml/min/cm², about 30 ml/min/cm², about 40 ml/min/cm², about 50 ml/min/cm², about 70 ml/min/cm², about 100 ml/min/cm², about 120 ml/min/cm², about 150 ml/min/cm², about 170 ml/min/cm², about 200 ml/min/cm², about 220 ml/min/cm², about 250 ml/min/cm², about 270 ml/min/cm², about 300 ml/min/cm², about 320 ml/min/cm², about 350 ml/min/cm², about 370 ml/min/cm², about 400 ml/min/cm², about 420 ml/min/cm², about 450 ml/min/cm², about 470 ml/min/cm², about 500 ml/min/cm², about 600 ml/min/cm², about 700 ml/min/cm², about 800 ml/min/cm², about 900 ml/min/cm², about 1,000 ml/min/cm², about 1,500 ml/min/cm², about 2,000 ml/min/cm², about 3,000 ml/min/cm², about 4,000 ml/min/cm², about 5,000 ml/min/cm², about 6,000 ml/min/cm², about 7,000 ml/min/cm², about 8,000 ml/min/cm², about 9,000 ml/min/cm², about 10,000 ml/min/cm², about 11,000 ml/min/cm², about 12,000 ml/min/cm², about 13,000 ml/min/cm², and about 14,000 ml/min/cm².

In still further aspects, the woven material can have a resiliently longitudinal stretchability from 0 to about 100 linear percent over its quiescent first length, including exemplary values of about 1, about 2, about 5, about 8, about 10, about 12, about 15, about 18, about 20, about 22, about 25, about 28, about 30, about 32, about 35, about 38, about 40, about 42, about 45, about 48, about 50, about 52, about 55, about 58, about 60, about 62, about 65, about 68, about 70, about 72, about 75, about 78, about about 82, about 85, about 88, about 90, about 92, about 95, and about 98 linear percent over its quiescent first length. In yet other aspects, the woven material can have a resiliently longitudinal stretchability of less than about 100 linear percent over its quiescent first length, including exemplary values of less than about 90, less than about less than about 70, less than about 60, less than about 50, less than about 40, less than about 30, less than about 30, less than about 20, and less than about 10 linear percent over its quiescent first length.

In still further aspects, the warp yarn and/or the weft yarn of the woven material can comprise a polyester, co-polyester, polyamide, polyolefin, polyaryletherketones, aromatic polymers, polyurethane, polytetrafluoroethylene, expanded polytetrafluorethylene, polyvinylidene fluoride, polyethers, polyureas, copolymers thereof, or a combination thereof. In yet other aspects, the warp yarn and/or the weft yarn of the woven material can comprise biocompatible thermoplastic polymers. Yet, in other aspects, the warp yarn and/or the weft yarn of the woven material can comprise biocompatible non-resorbable polymers. Yet, in some other examples, the warp yarn and/or the weft yarn can comprise a polyethylene terephthalate (PET), polytetrafluoroethylene (PTFE), Nylon, UHMWPE, PEEK, Liquid Crystalline Polymer, thermoplastic polyurethane (TPU), or a combination thereof. Still further, any other suitable natural or synthetic fibers or any combination thereof can be used. In still further aspects, the woven material can be material as disclosed above for the use of exemplary inner skirt 16.

In still further aspects, and as disclosed above, the second material is a knitted material. The knitted material can be a weft-knitted material in some aspects, while it can be a warp knitted material in other aspects. In yet still further aspects, the knitted material can comprise crochet knit and/or warp-knit fabric.

In certain aspects, the knitted material can comprise a pile yarn. In such aspects, the pile yarns can be arranged to form a looped pile. In some aspects, the pile yarn can also be cut to form a cut pile. In some aspects, the pile yarn can comprise a flat or textured yarn. In yet further aspects, the pile yarn can comprise a combination of the flat and textured yarn.

In some aspects, the pile yarn can have a size from about 5 denier to about 200 denier, including exemplary values of about 7 denier, about 10 denier, about 12 denier, about 15 denier, about 20 denier, about 22 denier, about 25 denier, about 30 denier, about 35 denier, about 40 denier, about 45 denier, about 50 denier, about 55 denier, about 60 denier, about 65 denier, about 70 denier, about 75 denier, about 80 denier, about 85 denier, about 90 denier, about 95 denier, about 100 denier, about 110 denier, about 120 denier, about 130 denier, about 140 denier, about 150 denier, about 160 denier, about 170 denier, about 180 denier, and about 190 denier. It is understood that the pile yarn can have any denier values that fall between any two foregoing values.

In still other aspects, the pile yarn can have a filament count from about 5 to about 200, including exemplary values of about 10, about 15, about 20, about 25, about 30, about 35, about 40, about 45, about 50, about 55, about 60, about 65, about 70, about 75, about 80, about 85, about 90, about 95, about 110, about 115, about 120, about 125, about 130, about 135, about 140, about 145, about 150, about 155, about 160, about 165, about 170, about 175, about 180, about 185, about 190, and about 195. In yet other aspects, the pile yarn can have a filament count higher than 200, higher than 250, or even higher than 300. It is understood that pile yarn can have a filament count value between any two foregoing values.

In still further aspects, the pile yarn can comprise biocompatible thermoplastic polymers. Yet, in other aspects, the pile yarn can comprise biocompatible non-resorbable polymers. In still further aspects, the yarn of the knitted material can comprise polyester, co-polyester, polyamide, polyolefin, polyaryletherketones, aromatic polymers, polyurethane, or any combination thereof. Yet, in some other examples, the warp yarn and/or the weft yarn can comprise a polyethylene terephthalate (PET), polytetrafluoroethylene (PTFE), Nylon, UHMWPE, PEEK, Liquid Crystalline Polymer, thermoplastic polyurethane (TPU), or a combination thereof. Still further, any other suitable natural or synthetic fibers or any combination thereof can be used.

In still further aspects, the knitted material can include velour, velvet, velveteen, corduroy, terrycloth, fleece, etc. The knitted material, having pile yarn, for example, has a much greater surface area than similarly sized skirts formed from flat or woven materials and therefore can enhance tissue ingrowth compared to known skirts. Promoting tissue growth into the knitted material can decrease perivalvular leakage, increase retention of the valve at the implant site and contribute to long-term stability of the valve. In some configurations, the surface area of the knitted material can be further increased by using textured yarns having an increased surface area due to, for example, a wavy or undulating structure. It is understood that the combination of the knitted and woven yarn both decreases PVL and reduces the crimped profile.

In still further aspects, the knitted material can have permeability from 0 to about 15,000 ml/min/cm² of water at a pressure of 120 mm of mercury, including exemplary values of about 5 ml/min/cm², about 10 ml/min/cm², about 20 ml/min/cm², about 30 ml/min/cm², about 40 ml/min/cm², about 50 ml/min/cm², about 70 ml/min/cm², about 100 ml/min/cm², about 150 ml/min/cm², about 200 ml/min/cm², about 300 ml/min/cm², about 400 ml/min/cm², about 500 ml/min/cm², about 600 ml/min/cm², about 700 ml/min/cm², about 800 ml/min/cm², about 900 ml/min/cm², about 1,000 ml/min/cm², about 1,500 ml/min/cm², about 2,000 ml/min/cm², about 3,000 ml/min/cm², about 4,000 ml/min/cm², about 5,000 ml/min/cm², about 6,000 ml/min/cm², about 7,000 ml/min/cm², about 8,000 ml/min/cm², about 9,000 ml/min/cm², about 10,000 ml/min/cm², about 11,000 ml/min/cm², about 12,000 ml/min/cm², about 13,000 ml/min/cm², and about 14,000 ml/min/cm².

In still further aspects, the knitted material can have a resiliently longitudinal stretchability from greater than 0 to about 300 linear percent over its quiescent first length, including exemplary values of about 1, about 2, about 5, about 8, about 10, about 12, about 15, about 18, about 20, about 22, about 25, about 28, about 30, about 32, about 35, about 38, about 40, about 42, about 45, about 48, about 50, about 52, about 55, about 58, about 60, about 62, about 65, about 68, about 70, about 72, about about 78, about 80, about 82, about 85, about 88, about 90, about 92, about 95, about 98, about 100, about 110, about 120, about 130, about 140, about 150, about 160, about 170, about 180, about 190, about 195, about 200, about 210, about 220, about 230, about 240, about 250, about 260, about 270, and about 280 linear percent over its quiescent first length. In yet other aspects, the knitted material can have a resiliently longitudinal stretchability of at least about 5 linear percent over its quiescent first length, including exemplary values of at least about 10, at least about 20, at least about 30, at least about 40 at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, and at least about 290 linear percent over its quiescent second length.

In some aspects, the first material can have a first length and a first width, wherein the first length is measured between a proximal end and a distal end of the first material. In yet other aspects, the second material can be defined by a second length and a second width, wherein the second length is measured between a proximal end and a distal end of the second material. It is understood that the second length can be the same or different from the first length. Similarly, there are also aspects where the second and the first width are the same or different.

In certain aspects, the first and/or the second widths are substantially identical to a circumference of the annular frame. Yet, in other aspects, the first and/or the second width are greater than the circumference of the annular frame such that when the textile is mounted around of the annular frame, the first and/or the second materials can form a complete covering of the desired portion of the annular frame without any tension in the textile material itself. It is also understood that in some aspects, the textile can be pre-formed prior to mounting it on the annular frame, or its width can be decided during the mounting on the annular frame to accommodate a desired configuration of the device. It is also understood that the width of textile material and particularly the first and/or second widths of the first and/or second materials can be configured to be adapted to both crimped and expanded profiles of the implantable medical device. It is understood that the widths of the first and the second materials are configured to allow the textile to snuggle the annular frame such that no unnecessary tension is created during implantation or use of the device.

In aspects, where for example, the second material superposes the first material, the width of the second material can be smaller than a circumference of the annular frame. In such exemplary and unlimiting aspects, the width of the first material can be substantially identical to a circumference of the annular frame.

In some aspects, the first length of the first material can be substantially identical to a length of the first portion of the annular frame. Yet, in still further aspects, the first length of the first material can be shorter than a length of the first portion of the annular frame. In yet further aspects, the first length of the first material can also be longer than a length of the first portion of the annular frame. In such exemplary aspects, if needed, the proximal portion of the first material can snug around the proximal portion of the annular frame and fully cover a proximal end of the frame.

Yet, in other aspects, the second length can be substantially identical to a length of the first portion of the annular frame. Yet, in still further aspects, the second length of the second material can be shorter than a length of the first portion of the annular frame. In yet further aspects, the second length of the second material can also be longer than a length of the first portion of the annular frame. In such exemplary aspects, if needed, the proximal portion of the second material can snug around the proximal portion of the annular frame and fully cover a proximal end of the frame.

In still further aspects, the first length of the first material is longer than the second length of the second material. In yet further aspects, the first length of the first material can be longer than a length of the first portion of the annular frame, while the second length of the second material is shorter than the first length of the first material. In other aspects, the second length of the second material can be substantially identical to a length of the first portion of the annular frame or shorter than a length of the first portion of the annular frame, or even larger than a length of the first portion of the annular frame as long it is shorter than the first length of the first material.

It is further understood that the first and/or the second materials can have any desired shape. For example, in some aspects, the first length of the first material can be substantially identical along the first width. In such aspects, the proximal and distal ends of the first material can be substantially straight along the width of the first material. Similar aspects can be applied to the second material. In such exemplary and unlimiting aspects, the second length of the second material can be substantially identical along the second width.

In some exemplary aspects, the proximal and/or distal end of the first material are substantially parallel to each other such that the first length is substantially identical along a circumference of the annular frame. While in other aspects, the proximal and/or distal end of the second material are substantially parallel to each other such that the second length is substantially identical along a circumference of the annular frame.

In still further aspects, the proximal and/or distal end of the first material can have a shape such that the first length varies along a circumference of the annular frame. While in still further aspects, the proximal and/or distal end of the second material has a shape such that the second length varies along a circumference of the annular frame.

In such exemplary aspects, the shape of the first and second materials can be the same or different. In yet other aspects, the shape of the proximal and/or distal end of the first and/or second materials can be random or repetitive. In yet still, further aspects, the shape of the proximal and/or distal end of the first and/or second materials can follow a pattern of at least a portion of the plurality of struts.

Yet, in other aspects, the first and/or second material can have any desired shape. For example, the first material can have substantially straight edges, while the second material can form a geometric shape. In such an aspect, the geometric shape of the second material can be defined by the desired application. It is understood that any geometric shapes that would allow decreasing crimped profile of the device while sustaining PVL sealing properties can be considered.

In certain aspects and as shown in FIG. 2, both distal and proximal ends of the first material and/or second material can be substantially straight. In still further exemplary aspects, and as shown in FIGS. 3 and 4, the distal and proximal ends of the first material 18b can be substantially straight, while the distal and proximal end of the second material 18a can form valleys and apexes. In yet other exemplary and unlimiting aspects, and as schematically shown in FIG. 13, the proximal end 160 of the textile can be substantially straight while the distal end 162 of the textile can define a plurality of alternating projections, or castellations, that generally follow the shape of a row of struts of the frame. In yet still other exemplary aspects, the proximal and distal ends 160, 162 can have other shapes. For example, in one implementation, the distal end can be formed with a plurality of projections generally conforming to the shape of a row of struts of the frame, while the proximal end can be straight (not shown).

It is understood that the first and second materials can be coupled to each other to form at least one joining region. It is further understood that the coupling can be performed by any known in the art methods. In some aspects, the first and the second materials can be coupled with a fastener, for example. In such exemplary and unlimiting aspects, the fasteners can comprise staples, sutures, adhesives, and the like. In yet other aspects, the coupling can be done through a heat-fusing bonding of the two materials. In yet other aspects, the coupling can be done through ultrasonic welding.

In still further aspects, this disclosure refers to any of the disclosed above implantable devices, where the first material and the second materials are coupled by an ultrasonic welding to form at least one joining region. In still further aspects, two or more joining regions can be present.

Now referring again to exemplary coupling configurations of the composite materials comprising the first and the second materials as shown in FIGS. 2-4. Again, it is understood that the configurations shown in the figures are only exemplary, and any other additional configurations can be constructed as needed.

FIG. 2 shows a configuration where the second material 18a, for example, a knitted material, is superposed on the first material 18b, for example, a woven material such that a least a portion of the first material is free of the second material and where the joining region 18c is formed by the ultrasonic welding. In this exemplary and unlimiting aspect, the joining region can extend along the width (circumference) of the second and the first materials coupled to each other. In other aspects (not shown), the composite material can have an additional joining region anywhere along the length and/or width of the composite material. For example, the additional joining region can be positioned along an edge where the first and the second materials are fully superposed. If more than one joining regions are present, in some aspects, such regions can be disposed randomly at the composite material. However, yet in other aspects, the two or more joining regions can be disposed in a predetermined pattern.

FIG. 3 shows an exemplary aspect where the second material 18a has a predetermined pattern and is disposed on the first material 18b. The joining regions 18c are formed along the edges of the second material to couple it to the first material. In such exemplary aspects, the predetermined pattern of the joining regions can follow a pattern of at least a portion of the plurality of struts of the annular frame.

A different exemplary configuration is shown in FIG. 4. In this configuration, the joining regions 18c are formed in a pattern along the second length and the second width of the second material. For example, in such exemplary and unlimiting aspects, the two or more joining regions can be arranged as a plurality of islands along a length of the composite material and along a circumference of the annular frame (FIG. 4). It is understood that in such exemplary and unlimiting aspects, each island of the plurality of islands has the same or different shape. It is further understood that the shape and shape's size can be selected such that the resulting textile material can balance various properties, and more specifically, it can provide for a substantially reduced crimped profile while still providing a substantial seal against PVL when compared to conventional valves.

It is understood that a thickness of the textile at the at least one of the joining regions is different from a thickness of the textile outside of the joining regions. In aspects where the ultrasonic welding is used to join the first and the second materials, the formed joining region has a smaller thickness than a thickness at other locations of the textile having both first and the second materials.

For example and without limitations, a thickness of the first material can be anywhere between 50 microns to about 100 microns, including exemplary values of about 60 microns, about 65 microns, about 70 microns, about 75 microns, about 80 microns, about 85 microns, about 90 microns, and about 95 microns. Yet in other aspects, a thickness of the second material is from about 0.05 mm to about 2.5 mm, including exemplary values of about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, and about 2.4 mm.

In still further aspects, the uncompressed thickness of the textile material comprising both the first material and the second material can be anywhere between about 0.5 mm to about 2.5 mm, including the exemplary values of about 6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, and about 2.4 mm. It is understood that the textile material can have any uncompressed thickness value between any two disclosed above values.

In yet further aspects, the compressed thickness (when the valve is crimped) of the textile material can be anywhere between about 0.1 mm to about 1 mm, including exemplary values of about mm, about 0.2 mm, about 0.3 mm, about 0.4 mm, about 0.5 mm, about 0.6 mm, about 0.7 mm, about 0.8 mm, and about 0.9 mm.

It is understood that a thickness of the joining regions, however, is lower than the overall thickness of the textile due to at least partial melting of the first and the second materials and forming a bond. However, it is further understood that while in some aspects, the thickness of the joining regions can be smaller than the thickness of the second material, in other aspects, the thickness of the joining regions can be larger than the thickness of the first material. The exact thickness of the joining regions can be controlled as desired by controlling the operating parameters of the ultrasonic welder. Similarly, the width and the length of the joining regions can be defined as desired.

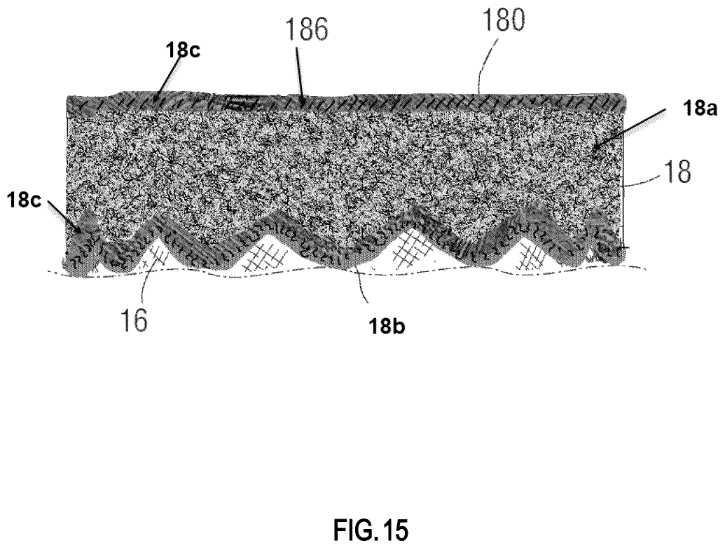

In still further aspects, the at least one joining region can be along the proximal end of the first portion of the annular frame. While in other aspects, the at least one joining region can be along the distal end of the first portion. An exemplary coupling of the outer skirt 18 to the frame and the inner skirt 16 is shown in FIG. 15. FIG. 15 shows one joining region disposed along the proximal end of the first portion of the annular frame and is connected to the frame with sutures 186. Yet, the distal end of the skirt has an additional joining region having a predetermined pattern and coupled to the frame and the inner skirt 16.

In certain aspects, where the at least one joining region is disposed along a proximal end of the textile material, the textile can be coupled to the proximal end of the annular frame through the joining region of the composite material (as shown in FIG. 15, for example). In such aspects, the textile can be coupled to the annular frame with one or more fasteners. Any known in the art fasteners can be used. In certain aspects, the one or more fasteners comprise at least one suture. In such aspects, any known in the art sutures can be used, for example, and without limitation, Ethibond Excel® PET suture (Johnson & Johnson, New Brunswick, N.J.), or PTFE sutures can be utilized.

In still further aspects, the textile can be coupled to the proximal end of the annular frame through portions of the composite material that don't have joining regions. Again in such aspects, the textile can be coupled to the annular frame with one or more fasteners, wherein the one or more fasteners can comprise at least one suture, such as Ethibond Excel® PET suture (Johnson & Johnson, New Brunswick, N.J.)

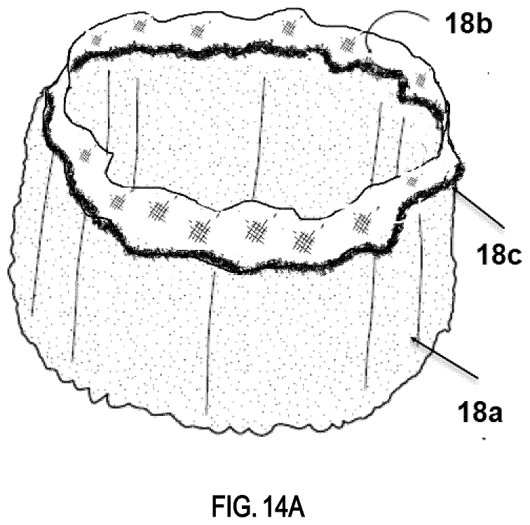

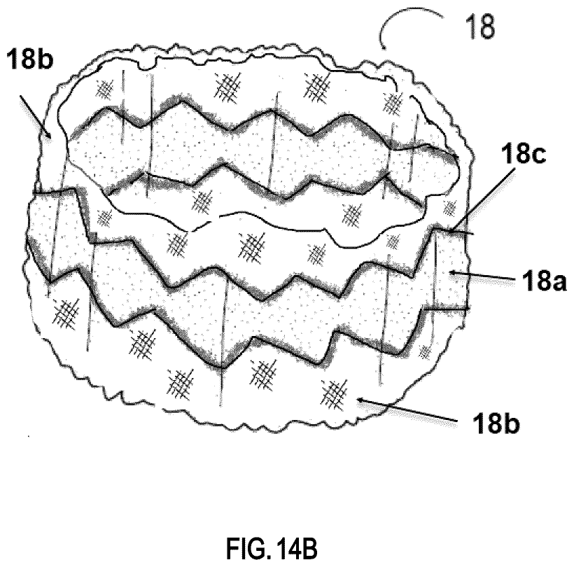

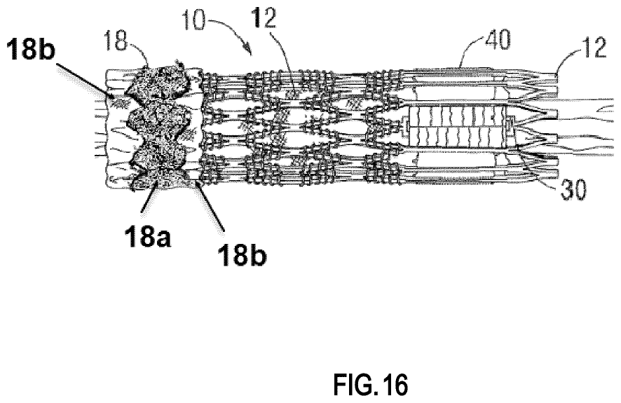

In still further exemplary and unlimiting aspects, the textile can further comprise at least a portion comprising the first material such that it is not overlaying with or is not overlaid by the second material. Such exemplary aspects are shown in FIGS. 14A-14B, where at least a portion of the first material 18b is not overlaid with the second material 18a. FIG. 16 shows the valve in a crimped configuration where some portion of the textile has the first material 18b that is not overlaid with the second material 18a.

Similarly, there are aspects where the textile comprises at least a portion comprising the second material such that it is not overlaying with or is not overlaid by the first material (not shown).

In the aspects where the at least a portion of the textile where the first material is overlaid with or overlaying at the second material present, such the at least portion can be disposed along the proximal and/or distal end of the textile (FIGS. 14A-14B).

In still further aspects, and as shown in FIG. 1 or FIG. 13, the surface of the annular frame has a second portion 1820 that is free of the textile, and wherein the second portion extends between the outflow end of the annular frame and the distal end of the first portion of the annular frame.

In some aspects and as disclosed above, the textile material disclosed herein is used as an outer skirt has a sealing function to prevent PVL and to secure the valve in place. In some aspects, since the outer skirt can be assembled on an expanded frame as the frame gets longer in length (radially compressed), a certain tension can be introduced along the length of the outer sealing member. However, it is further understood that such tension is lower than in any other reference device where the outer member has a length shorter than the length of the first portion.

Exemplary schematic of the outer sealing member assembled on the annular frame in various configurations is also shown in FIG. 13 in the expanded state or in FIG. 16 in the crimped state.

A change in the length of the outer sealing member and the first portion of the annular frame can be observed when the annular frame changes from compressed to the expanded configuration.

In still further aspects, the outer skirt can be laser cut or ultrasonic cut from the pre-made textile materials or can be assembled on the frame.

The outer skirt comprising the composite material described herein can also contribute to improved compressibility and shape memory properties of the outer skirt over known valve coverings and skirts. For example, the knitted material can be compliant such that it compresses under load (e.g., when in contact with tissue, other implants, or the like) and returns to its original size and shape when the load is relieved. This can help to improve sealing between the outer skirt and the tissue of the native annulus or a surrounding support structure in which the prosthetic valve is deployed. Aspects of an implantable support structure that is adapted to receive a prosthetic valve and retain it within the native mitral valve are disclosed in co-pending Application No. 62/449,320, filed Jan. 23, 2017, and application Ser. No. 15/876,053, filed Jan. 19, 2018, which are incorporated herein by reference. The compressibility provided by the knitted material 18a of the outer skirt 18 is also beneficial in reducing the crimped profile of the valve. Additionally, the outer skirt 18 can prevent the leaflets 14 or portions thereof from extending through spaces between the struts of the frame 12 as the prosthetic valve is crimped, thereby protecting against damage to the leaflets due to pinching of the leaflets between struts.

As discussed above, various techniques and configurations can be used to secure the outer skirt 18 to frame 12 and/or the inner skirt 16. In certain aspects, the outer sealing member 18 can be coupled to the proximal end 1806a and the distal end 1806b of the first portion by a fastener, as shown for example in FIG. 15. In still further aspects, a fastener can be used to couple the outer skirt 18 to the inner skirt 16. It is understood that the fasteners can comprise any fasteners known in the art. For example, and without limitations, the fasteners can comprise sutures, pins, rivets, ultrasonic welding, laser welding, adhesive bonding, or any combination thereof.

In some aspects, the outer skirt 18 can be attached to the frame 12 when the frame is in the radially compressed configuration, and the outer skirt 18 is straightened along the outer surface of the first portion of the compressed annular frame (FIG. 16).

In such exemplary aspects, a lower edge portion of the inner skirt 16 can be wrapped around the inflow end 15 of the frame 12, and the lower edge portion of the outer skirt 18 can be attached to the lower edge portion 180 of the inner skirt 16 and/or the frame 12, such as with a fastener that comprises one or more sutures or stitches 182 (as best shown in FIG. 15) and/or an adhesive. In some aspects, the attachment can be done through the joining region between the first material and the second material 18c. In lieu of or in addition to sutures, the outer skirt 18 can be attached to the inner skirt 16, for example, by ultrasonic welding. In other aspects, the lower edge portion 180 of the inner skirt 16 can be wrapped around the inflow end 15 of the annular frame and extend between the outer surface of the frame and the outer skirt 18 (i.e., the outer skirt 18 is radially outward of the lower edge portion 180 of the inner skirt 16).

The outer skirt can be attached in the distal end of the first portion to the rows of struts of the annular frame as described below. For example, as shown in FIG. 1, each projection 164 of the outer skirt 18 can be attached to the third row III of struts 26 (FIG. 5) of the frame 12. The projections 164 can, for example, be wrapped over respective struts 26 of row III and secured with sutures 184. The outer skirt 18 can be further secured to the frame 12 by suturing an intermediate portion of the outer skirt (a portion between the proximal end and distal end) to struts of the frame, such as struts 24 of the second row II of struts.

The prosthetic valve 10 can be configured for and mounted on a suitable delivery apparatus for implantation in a subject. Several catheter-based delivery apparatuses are known; a non-limiting example of a suitable catheter-based delivery apparatus includes that disclosed in U.S. Patent Application Publication No. 2013/0030519, which is incorporated by reference herein in its entirety, and U.S. Patent Application Publication No. 2012/0123529.

To implant a plastically-expandable prosthetic valve 10 within a patient, the prosthetic valve 10, including the outer skirt 18, can be crimped on an elongated shaft of a delivery apparatus. It is understood that in crimped (compressed configuration), the outer skirt 18 is snugly fit around the circumference of the annular frame without creating a substantial tension within the outer skirt fabric. It is understood, however, that in some aspects, the outer skirt can be assembled on an expanded frame as the frame gets longer in length (radially compressed).

The prosthetic valve, together with the delivery apparatus, can form a delivery assembly for implanting the prosthetic valve 10 in a patient's body. The shaft can comprise an inflatable balloon for expanding the prosthetic valve within the body. With the balloon deflated, the prosthetic valve 10 can then be percutaneously delivered to the desired implantation location (e.g., a native aortic valve region). Once prosthetic valve 10 is delivered to the implantation site (e.g., the native aortic valve) inside the body, the prosthetic valve 10 can be radially expanded to its functional state by inflating the balloon or equivalent expansion mechanism.

Alternatively, a self-expanding prosthetic valve 10 can be crimped to a radially collapsed configuration and restrained in the collapsed configuration by inserting the prosthetic valve 10, including the outer skirt 18, into a sheath or equivalent mechanism of a delivery catheter. The prosthetic valve 10 can then be percutaneously delivered to the desired implantation location. Once inside the body, the prosthetic valve 10 can be advanced from the delivery sheath, which allows the prosthetic valve to expand to its functional state.

### Methods

The present disclosure also provides for a method of forming an implantable medical device comprising: a) providing an annular frame having an inflow end, an outflow end, and a longitudinal axis and comprising a plurality of struts; b) circumferentially mounting a textile defined by a first surface and an opposite second surface, having a longitudinal axis and a transverse axis, wherein the longitudinal axis of the textile is substantially parallel to the longitudinal axis of the annular frame; and wherein the textile has a proximal end and a distal end and is mounted circumferentially around a first portion of the annular frame, wherein the first portion has a proximal end and a distal end, wherein the proximal end of the first portion is at the inflow end of the annular frame; wherein at least a portion of the textile comprises at least one composite material comprising a first material and a second material coupled together such that a crimp profile of the implantable medical device is reduced when compared to a substantially identical reference implantable medical device in the absence of the at least one composite material; wherein the first material exhibits a flexibility and stretchability lower than a flexibility and stretchability of the second material; and wherein the first material has higher a mechanical strength than a mechanical strength of the second material; and wherein the implantable medical device is configured to radially expand to an expanded configuration from the crimp profile.

In still further aspects, the proximal end of the textile material is coupled to the proximal end of the first portion of the annular frame. In further aspects, the distal end of the textile is coupled to the distal end of the first portion of the annular frame.

Any of the disclosed above textile materials can be utilized in the disclosed methods. In some methods, the first material is a woven material, while the second material is knitted material. Again, any of the disclosed above woven and knitted materials can be used.

In certain aspects, the textile can be used as an inner skirt. While in other aspects, the textile can be used as an outer skirt. In yet still further aspects, the textile can be used as the inner and outer skirts simultaneously. In such aspects, the frame is first disposed between the first and the second materials, and then the first and the second materials can be coupled to each other by any of the disclosed herein methods.

In still further aspects, when the first and the second materials are coupled together, at least one joining region can be formed. As disclosed above, any coupling methods can be utilized. For example, coupling methods can comprise the use of adhesives, sutures, heat-fusing, or ultrasonic welding. In the aspects where the ultrasonic welding is used, the ultrasonic welding is performed under conditions effective to form the at least one joining region having a predetermined thickness. In yet other aspects, the ultrasonic welding is performed under conditions effective to form the at least one joining region having a predetermined width.

It is understood that the conditions effective to form the at least one joining region having a predetermined thickness and/or width can comprise the use of a predetermined frequency, a predetermined weld time, a predetermined pressure applied to the textile material, hold time, and the like.

In certain preferred embodiments, the textile described herein can be used as an outer skirt to form the valve. Similarly, any of the methods disclosed above of attachment of the outer skirt to the annular frame can be utilized.

In still further aspects, the methods disclosed herein can comprise a step of impregnating any of the disclosed herein textile materials with a pharmaceutically active agent depending on the desired application. In still further aspects, the methods disclosed herein can comprise a step of coating any of the disclosed herein textile materials with any known in the art materials that can provide for any additional desired properties.

### EXEMPLARY ASPECTS

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

EXAMPLE 1: An implantable medical device comprising: an annular frame having an inflow end, an outflow end, and a longitudinal axis and comprising a plurality of struts; and a textile defined by a first surface and an opposite second surface, having a longitudinal axis and a transverse axis, wherein the longitudinal axis of the textile is substantially parallel to the longitudinal axis of the annular frame; and wherein the textile has a proximal end and a distal end and is mounted circumferentially around a first portion of the annular frame, wherein the first portion has a proximal end and a distal end, wherein the proximal end of the first portion is at the inflow end of the annular frame; wherein at least a portion of the textile comprises at least one composite material comprising a first material and a second material coupled together such that a crimped profile of the implantable medical device is reduced when compared to a substantially identical reference implantable medical device in the absence of the at least one composite material; wherein the first material exhibits a flexibility and stretchability lower than a flexibility and stretchability of the second material; wherein the first material has higher a mechanical strength than a mechanical strength of the second material; and wherein the implantable medical device is configured to radially expand to an expanded configuration from the crimped profile.

EXAMPLE 2: The implantable medical device of any examples herein, particularly example 1, wherein the proximal end of the textile is coupled to the proximal end of the first portion of the annular frame, and the distal end of the textile is coupled to the distal end of the first portion of the annular frame.

EXAMPLE 3: The implantable medical device of any examples herein, particularly example 1 or 2, wherein the first material and the second material are at least partially superposed.

EXAMPLE 4: The implantable medical device of any examples herein, particularly examples 1-3, wherein the first material is a woven material.

EXAMPLE 5: The implantable medical device of any examples herein, particularly examples 1-4, wherein the second material is a knitted material.

EXAMPLE 6: The implantable medical device of any examples herein, particularly examples 1-5, wherein the second material is at least partially superposed on the first material.

EXAMPLE 7: The implantable medical device of any examples herein, particularly examples 1-6, wherein the first material has a first length and a first width, wherein the first length is measured between a proximal end and a distal end of the first material.

EXAMPLE 8: The implantable medical device of any examples herein, particularly examples 1-7, wherein the second material has a second length and a second width, wherein the second length is measured between a proximal end and a distal end of the second material.

EXAMPLE 9: The implantable medical device of any examples herein, particularly example 8, wherein the second length is the same or different from the first length.

EXAMPLE 10: The implantable medical device of any examples herein, particularly example 8 or 9, wherein the second width is the same or different from the first width.

EXAMPLE 11: The implantable medical device of any examples herein, particularly examples 7-11, wherein the first width is substantially identical to a circumference of the annular frame.

EXAMPLE 12: The implantable medical device of any examples herein, particularly examples 8-11, wherein the second width is substantially identical to a circumference of the annular frame.

EXAMPLE 13: The implantable medical device of any examples herein, particularly examples 8-11, wherein the second width is smaller than a circumference of the annular frame.

EXAMPLE 14: The implantable medical device of any examples herein, particularly examples 7-13, wherein the first length is substantially identical to a length of the first portion of the annular frame.

EXAMPLE 15: The implantable medical device of any examples herein, particularly examples 8-14, wherein the second length is substantially identical to a length of the first portion of the annular frame.

EXAMPLE 16: The implantable medical device of any examples herein, particularly examples 7-13 or 15, wherein the first length is shorter than a length of the first portion of the annular frame.

EXAMPLE 17: The implantable medical device of any examples herein, particularly examples 8-14 or 16, wherein the second length is shorter than a length of the first portion of the annular frame.

EXAMPLE 18: The implantable medical device of any examples herein, particularly examples 7-13 or 15 or 17, wherein the first length is longer than a length of the first portion of the annular frame.

EXAMPLE 19: The implantable medical device of any examples herein, particularly examples 8-14 or 16 or 18, wherein the second length is longer than a length of the first portion of the annular frame.

EXAMPLE 20: The implantable medical device of any examples herein, particularly examples 1-19, wherein the first material and the second material are coupled by an ultrasonic welding such that at least one joining region is formed.

EXAMPLE 21: The implantable medical device of any examples herein, particularly example 20, wherein two or more joining regions are present.

EXAMPLE 22: The implantable medical device of any examples herein, particularly example 21, wherein the two or more joining regions are disposed randomly.

EXAMPLE 23: The implantable medical device of any examples herein, particularly example 22, wherein the two or more joining regions are disposed in a predetermined pattern.

EXAMPLE 24: The implantable medical device of any examples herein, particularly examples 20-23, wherein a thickness of the textile at the at least one of joining regions is different from a thickness of the textile outside of the joining regions.

EXAMPLE 25: The implantable medical device of any examples herein, particularly examples 23-24, wherein the predetermined pattern follows a pattern of at least a portion of the plurality of struts.

EXAMPLE 26: The implantable medical device of any examples herein, particularly examples 21-25, wherein the two or more joining regions are arranged as a plurality of islands along a length of the composite material and along a circumference of the annular frame.

EXAMPLE 27: The implantable medical device of any examples herein, particularly example 26, wherein each island of the plurality of islands has the same or different shape.

EXAMPLE 28: The implantable medical device of any examples herein, particularly examples 21-26, wherein the two or more joining regions are arranged such that the crimped profile of the implantable medical device is reduced when compared to a substantially identical reference implantable medical device in the absence of the at least one composite material.

EXAMPLE 29: The implantable medical device of any examples herein, particularly examples 20-28, wherein the at least one joining region is positioned along the proximal end of the first portion of the annular frame.

EXAMPLE 30: The implantable medical device of any examples herein, particularly examples 20-29, wherein the at least one joining region is positioned along the distal end of the first portion of the annular frame.

EXAMPLE 31: The implantable medical device of any examples herein, particularly examples 29-30, wherein the textile is coupled to the proximal end of the first portion of the annular frame through the joining region of the composite material.

EXAMPLE 32: The implantable medical device of any examples herein, particularly example 31, wherein the textile is coupled to the annular frame with one or more fasteners.

EXAMPLE 33: The implantable medical device of any examples herein, particularly example 32, wherein the one or more fasteners comprise at least one suture.

EXAMPLE 34: The implantable medical device of any examples herein, particularly examples 30-33, wherein the textile is coupled to the distal end of the first portion of the annular frame through the joining region of the composite material.

EXAMPLE 35: The implantable medical device of any examples herein, particularly example 34 wherein the textile is coupled to the annular frame with one or more fasteners.

EXAMPLE 36: The implantable medical device of any examples herein, particularly example 35, wherein the one or more fasteners comprise at least one suture.

EXAMPLE 37: The implantable medical device of any examples herein, particularly examples 1-36, wherein the textile further comprises at least a portion where the first and the second materials do not overlay each other.

EXAMPLE 38: The implantable medical device of any examples herein, particularly example 37, wherein the at least portion of the textile where the first and the second materials do not overlay each other is disposed along the proximal and/or distal end of the textile.

EXAMPLE 39: The implantable medical device of any examples herein, particularly examples 6-38, wherein the proximal and distal end of the first material are substantially parallel to each other such that the first length is substantially identical along a circumference of the annular frame.

EXAMPLE 40: The implantable medical device of any examples herein, particularly examples 8-39, wherein the proximal and distal end of the second material are substantially parallel to each other such that the second length is substantially identical along a circumference of the annular frame.

EXAMPLE 41: implantable medical device of any examples herein, particularly examples 7-40, wherein the proximal and/or distal end of the first material has a shape such that the first length varies along a circumference of the annular frame.

EXAMPLE 42: The implantable medical device of any examples herein, particularly examples 8-41, wherein the proximal and/or distal end of the second material has a shape such that the second length varies along a circumference of the annular frame.

EXAMPLE 43: The implantable medical device of any examples herein, particularly example 41 or 42, wherein the shape of the proximal and/or distal end of the first and/or second materials is random or repetitive.

EXAMPLE 44: The implantable medical device of any examples herein, particularly examples 41-43, wherein the shape of the proximal and/or distal end of the first and/or second materials follows a pattern of at least a portion of the plurality of struts.

EXAMPLE 45: The implantable medical device of any examples herein, particularly examples 4-44, wherein the woven material comprises a plurality of warp and weft yarns.

EXAMPLE 46: The implantable medical device of any examples herein, particularly example 45, wherein the warp yarn and/or weft yarn are multifilament yarns and comprise a fully-drawn yarn, spin-drawn yarn, low or not twisted yarn, twisted yarn, a flat yarn, a textured yarn, or any combination thereof.

EXAMPLE 47: The implantable medical device of any examples herein, particularly example 46, wherein the warp yarn and/or weft yarn has a size from about 5 denier to about 200 denier and a filament count from about 5 to about 200.

EXAMPLE 48: The implantable medical device of any examples herein, particularly examples 4-47, wherein the woven material has a tenacity from about 20 to about 500 cN/tex.

EXAMPLE 49: The implantable medical device of any examples herein, particularly examples 4-48, where the woven material has permeability from 0 to about 15,000 ml/min/cm² of water at a pressure of 120 mm of mercury.

EXAMPLE 50: The implantable medical device of any examples herein, particularly examples 4-49, where the woven material has a resiliently longitudinal stretchability from 0 to about 100 linear percent over its quiescent first length.

EXAMPLE 51: The implantable medical device of any examples herein, particularly examples 5-50, where the knitted material has permeability from about 0 to about 15,000 ml/min/cm² of water at a pressure of 120 mm of mercury.

EXAMPLE 52: The implantable medical device of any examples herein, particularly examples 5-51, where the woven material has a resiliently longitudinal stretchability from greater than 0 to about 300 linear percent over its quiescent second length.

EXAMPLE 53: The implantable medical device of any examples herein, particularly examples 45-52, wherein the warp yarn and/or weft yarn comprises a polyester, co-polyester, polyamide, polyolefin, polyaryletherketones, aromatic polymers, polyurethane, polytetrafluoroethylene, expanded polytetrafluorethylene, polyvinylidene fluoride, polyethers, polyureas, copolymers thereof, or a combination thereof.

EXAMPLE 54: The implantable medical device of any examples herein, particularly examples 45-53, wherein the warp yarn and/or weft yarn comprises polyethylene terephthalate (PET), polytetrafluoroethylene (PTFE), Nylon, UHMWPE, PEEK, Liquid Crystalline Polymer, thermoplastic polyurethane (TPU), or a combination thereof.

EXAMPLE 55: The implantable medical device of any examples herein, particularly examples 5-54, wherein the knitted material comprises crochet knit and/or warp-knit fabric.

EXAMPLE 56: The implantable medical device of any examples herein, particularly examples 5-55, wherein the knitted material comprises a pile yarn.

EXAMPLE 57: The implantable medical device of any examples herein, particularly example 56, wherein the pile yarn comprises a flat yarn, a textured yarn, or a combination thereof.

EXAMPLE 58: The implantable medical device of any examples herein, particularly example 56 or 57, wherein the pile yarn comprises a polyester, co-polyester, polyamide, polyolefin, polyaryletherketones, aromatic polymers, polyurethane, polytetrafluoroethylene, expanded polytetrafluorethylene, polyvinylidene fluoride, polyethers, polyureas, copolymers thereof, or a combination thereof.

EXAMPLE 59 The implantable medical device of any examples herein, particularly examples 56-58, wherein the pile yarn comprises polyethylene terephthalate (PET), polytetrafluoroethylene (PTFE), Nylon, UHMWPE, PEEK, Liquid Crystalline Polymer, thermoplastic polyurethane (TPU) or a combination thereof.

EXAMPLE 60: The implantable medical device of any examples herein, particularly examples 1-59, wherein the surface of the annular frame has a second portion that is free of the textile and wherein the second portion extends between the outflow end of the annular frame and the distal end of the first portion of the annular frame.

EXAMPLE 61: The implantable medical device of any examples herein, particularly examples 1-60, wherein the textile is disposed on an outer surface of the annular frame and is an outer skirt.

EXAMPLE 62: The implantable medical device of any examples herein, particularly example 61, wherein the outer skirt is a sealing member configured to prevent a paravalvular leak.

EXAMPLE 63: The implantable medical device of any examples herein, particularly examples 1-62, wherein the implantable medical device is a heart valve.

EXAMPLE 64: A method of forming an implantable medical device comprising: a) providing an annular frame having an inflow end, an outflow end, and a longitudinal axis and comprising a plurality of struts; b) circumferentially mounting a textile defined by a first surface and an opposite second surface, having a longitudinal axis and a transverse axis, wherein the longitudinal axis of the textile is substantially parallel to the longitudinal axis of the annular frame; and wherein the textile has a proximal end and a distal end and is mounted circumferentially around a first portion of the annular frame, wherein the first portion has a proximal end and a distal end, wherein the proximal end of the first portion is at the inflow end of the annular frame; wherein at least a portion of the textile comprises at least one composite material comprising a first material and a second material coupled together such that a crimped profile of the implantable medical device is reduced when compared to a substantially identical reference implantable medical device in the absence of the at least one composite material; wherein the first material exhibits a flexibility and stretchability lower than a flexibility and stretchability of the second material; and wherein the first material has higher a mechanical strength than a mechanical strength of the second material, and wherein the implantable medical device is configured to radially expand to an expanded configuration from the crimped profile.

EXAMPLE 65: The method of any examples herein, particularly example 64, comprising coupling the proximal end of the textile to the proximal end of the first portion of the annular frame and coupling the distal end of the textile to the distal end of the first portion of the annular frame.

EXAMPLE 66: The method of any examples herein, particularly example 64 or 65, wherein the first material and the second material are at least partially superposed.

EXAMPLE 67: The method of any examples herein, particularly examples 64-66, wherein the first material is a woven material.

EXAMPLE 68: The method of any examples herein, particularly examples 64-67, wherein the second material is a knitted material.

EXAMPLE 69: The method of any examples herein, particularly examples 64-68, wherein the second material is superposed on the first material.

EXAMPLE 70: The method of any examples herein, particularly examples 64-69, wherein the first material has a first length and a first width, wherein the first length is measured between a proximal end and a distal end of the first material.

EXAMPLE 71: The method of any examples herein, particularly examples 64-70, wherein the second material has a second length and a second width, wherein the second length is measured between a proximal end and a distal end of the second material.

EXAMPLE 72: The method of any examples herein, particularly example 71, wherein the second length is the same or different from the first length.

EXAMPLE 73: The method of any examples herein, particularly example 71 or 72, wherein the second width is the same or different from the first width.

EXAMPLE 74: The method of any examples herein, particularly examples 70-73, wherein the first width is substantially identical to a circumference of the annular frame.

EXAMPLE 75: The method of any examples herein, particularly examples 71-74, wherein the second width is substantially identical to a circumference of the annular frame.

EXAMPLE 76: The method of any examples herein, particularly examples 71-75, wherein the second width is smaller than a circumference of the annular frame.

EXAMPLE 77: The method of any examples herein, particularly examples 71-76, wherein the first length is substantially identical to a length of the first portion of the annular frame.

EXAMPLE 78: The method of any examples herein, particularly examples 71-77, wherein the second length is substantially identical to a length of the first portion of the annular frame.

EXAMPLE 79: The method of any examples herein, particularly examples 70-76 or 78, wherein the first length is shorter than a length of the first portion of the annular frame.

EXAMPLE 80: The method of any examples herein, particularly examples 72-78 or 80, wherein the second length is shorter than a length of the first portion of the annular frame.

EXAMPLE 81: The method of any examples herein, particularly examples 71-76 or 78 or 80, wherein the first length is longer than a length of the first portion of the annular frame.

EXAMPLE 82: The method of any examples herein, particularly examples 71-77 or 79 or 81, wherein the second length is longer than a length of the first portion of the annular frame.

EXAMPLE 83: The method of any examples herein, particularly examples 1-83, wherein the first material and the second material are coupled by an ultrasonic welding such that at least one joining region is formed.

EXAMPLE 84: The method of any examples herein, particularly example 83, wherein the ultrasonic welding is performed under conditions effective to form the at least one joining region having a predetermined thickness.

EXAMPLE 85: The method of any examples herein, particularly example 83 or 84, wherein the ultrasonic welding is performed under conditions effective to form the at least one joining region having a predetermined width.

EXAMPLE 86: The method of any examples herein, particularly examples 83-85, wherein the ultrasonic welding is performed under conditions effective to form the at least one joining region having a predetermined pattern.

EXAMPLE 87: The method of any examples herein, particularly examples 83-86, wherein two or more joining regions are present.

EXAMPLE 88: The method of any examples herein, particularly example 87, wherein the two or more joining regions are disposed randomly.

EXAMPLE 89: The method of any examples herein, particularly example 88, wherein the two or more joining regions are disposed in a predetermined pattern.

EXAMPLE 90: The method of any examples herein, particularly examples 83-89, wherein a thickness of the textile at the at least one of joining regions is different from a thickness of the textile outside of the joining regions.

EXAMPLE 91: The method of any examples herein, particularly examples 87-90, wherein the predetermined pattern follows a pattern of at least a portion of the plurality of struts.

EXAMPLE 92: The method of any examples herein, particularly examples 87-91, wherein the two or more joining regions are arranged as a plurality of islands along a length of the composite material and along a circumference of the annular frame.

EXAMPLE 93: The method of any examples herein, particularly example 92, wherein each island of the plurality of islands has the same or different shape.

EXAMPLE 94: The method of any examples herein, particularly examples 87-93, wherein the two or more joining regions are arranged such that the crimped profile of the implantable medical device is reduced when compared to a substantially identical reference implantable medical device in the absence of the at least one composite material.

EXAMPLE 95: The method of any examples herein, particularly examples 83-94, wherein the at least one joining region is along the proximal end of the first portion of the annular frame.

EXAMPLE 96: The method of any examples herein, particularly examples 83-95, wherein the at least one joining region is along the distal end of the first portion of the annular frame.

EXAMPLE 97: The method of any examples herein, particularly examples 95-96, wherein the textile is coupled to the proximal end of the annular frame through the joining region of the composite material.

EXAMPLE 98: The method of any examples herein, particularly example 97, wherein the textile is coupled to the annular frame with one or more fasteners.

EXAMPLE 99: The method of any examples herein, particularly example 98, wherein the one or more fasteners comprise at least one suture.

EXAMPLE 100: The method of any examples herein, particularly examples 96-99, wherein the textile is coupled to the distal end of the first portion of the annular frame through the joining region of the composite material.

EXAMPLE 101: The method of any examples herein, particularly example 100, wherein the textile is coupled to the annular frame with one or more fasteners.

EXAMPLE 102: The method of any examples herein, particularly example 101, wherein the one or more fasteners comprise at least one suture.

EXAMPLE 103: The method of any examples herein, particularly examples 65-102, wherein the textile further comprises at least a portion where the first and the second materials do not overlay each other.

EXAMPLE 104: The method of any examples herein, particularly example 103, wherein the at least portion of the textile where the first and the second materials do not overlay each other is disposed along the proximal and/or distal end of the textile.

EXAMPLE 105: The method of any examples herein, particularly examples 70-104, wherein the proximal and distal end of the first material are substantially parallel to each other such that the first length is substantially identical along a circumference of the annular frame.

EXAMPLE 106: The method of any examples herein, particularly examples 71-105, wherein the proximal and distal end of the second material are substantially parallel to each other such that the second length is substantially identical along a circumference of the annular frame.

EXAMPLE 107: The method of any examples herein, particularly examples 70-106, wherein the proximal and/or distal end of the first material has a shape such that the first length varies along a circumference of the annular frame.

EXAMPLE 108: The method of any examples herein, particularly examples 71-107, wherein the proximal and/or distal end of the second material has a shape such that the first length varies along a circumference of the annular frame.

EXAMPLE 109: The method of any examples herein, particularly example 107 or 108, wherein the shape is random or repetitive along the proximal and/or distal edge.

EXAMPLE 110: The method of any examples herein, particularly examples 107-109, wherein the shape of the proximal and/or distal edge follows a pattern of at least a portion of the plurality of struts.

EXAMPLE 111: The method of any examples herein, particularly examples 67-110, wherein the woven material comprises a plurality of warp and weft yarns.

EXAMPLE 112: The method of any examples herein, particularly example 113, wherein the warp yarn and/or weft yarn are multifilament yarns and comprise a fully-drawn yarn, spin-drawn yarn, low or not twisted yarn, twisted yarn, a flat yarn, a textured yarn, or any combination thereof.

EXAMPLE 113: The method of any examples herein, particularly example 112, wherein the warp yarn and/or weft yarn has a size from about 5 denier to about 200 denier and a filament count from about 5 to about 200.

EXAMPLE 114: The method of any examples herein, particularly examples 67-113, wherein the woven material has a tenacity from about 20 to about 500 cN/tex.

EXAMPLE 115: The method of any examples herein, particularly examples 67-114, where the woven material has permeability from 0 to about 15,000 ml/min/cm² of water at a pressure of 120 mm of mercury.

EXAMPLE 116: The method of any examples herein, particularly examples 67-115, where the woven material has a resiliently longitudinal stretchability from 0 to about 100 linear percent over its quiescent first length.

EXAMPLE 117: The method of any examples herein, particularly examples 68-116, where the knitted material has permeability from 0 to about 15,000 ml/min/cm² of water at a pressure of 120 mm of mercury.

EXAMPLE 118: The method of any examples herein, particularly examples 68-117, where the woven material has a resiliently longitudinal stretchability from greater than 0 to about 300 linear percent over its quiescent second length.

EXAMPLE 119: The method of any examples herein, particularly examples 111-118, wherein the warp yarn and/or weft yarn comprises a polyester, co-polyester, polyamide, polyolefin, polyaryletherketones, aromatic polymers, polyurethane, polytetrafluoroethylene, expanded polytetrafluorethylene, polyvinylidene fluoride, polyethers, polyureas, copolymers thereof, or a combination thereof.

EXAMPLE 120: The method of any examples herein, particularly examples 111-119, wherein the warp yarn and/or weft yarn comprises polyethylene terephthalate (PET), polytetrafluoroethylene (PTFE), Nylon, UHMWPE, PEEK, Liquid Crystalline Polymer, thermoplastic polyurethane (TPU), or a combination thereof.

EXAMPLE 121: The method of any examples herein, particularly examples 68-120, wherein the knitted material comprises crochet knit and/or warp-knit fabric.

EXAMPLE 122: The method of any examples herein, particularly examples 68-121, wherein the knitted material comprises a pile yarn.

EXAMPLE 123: The method of any examples herein, particularly example 122, wherein the pile yarn comprises a flat, textured yarn, or a combination thereof.

EXAMPLE 1026: The method of any examples herein, particularly example 122 or 123, wherein the pile yarn comprises a polyester, co-polyester, polyamide, polyolefin, polyaryletherketones, aromatic polymers, polyurethane, polytetrafluoroethylene, expanded polytetrafluorethylene, polyvinylidene fluoride, polyethers, polyureas, copolymers thereof, or a combination thereof.

EXAMPLE 125: The method of any examples herein, particularly examples 122-124, wherein the pile yarn comprises polyethylene terephthalate (PET), polytetrafluoroethylene (PTFE), Nylon, UHMWPE, PEEK, Liquid Crystalline Polymer, thermoplastic polyurethane (TPU) or a combination thereof.

EXAMPLE 126: The method of any examples herein, particularly examples 64-125, wherein the surface of the annular frame has a second portion that is free of the textile and wherein the second portion extends between the outflow end of the annular frame and the distal end of the first portion of the annular frame.

EXAMPLE 127: The method of any examples herein, particularly examples 64-126, wherein the textile is disposed on an outer surface of the annular frame and is an outer skirt.

EXAMPLE 128: The method of any examples herein, particularly example 127, wherein the outer skirt is a sealing member configured to prevent a paravalvular leak.

EXAMPLE 129: The method of any examples herein, particularly examples 65-128, wherein the implantable medical device is a heart valve.

Although several aspects of the invention have been disclosed in the foregoing specification, it is understood by those skilled in the art that many modifications and other aspects of the invention will come to mind to which the invention pertains, having the benefit of the teaching presented in the foregoing description and associated drawings. It is thus understood that the invention is not limited to the specific aspects disclosed hereinabove and that many modifications and other aspects are intended to be included within the scope of the appended claims. Moreover, although specific terms are employed herein, as well as in the claims which follow, they are used only in a generic and descriptive sense and not for the purposes of limiting the described invention nor the claims which follow. We, therefore, claim as our invention all that comes within the scope and spirit of these claims.

We claim:

1. An implantable medical device comprising:
an annular frame having an inflow end, an outflow end, and a longitudinal axis and comprising a plurality of struts; and
a textile defined by a first surface and an opposite second surface, having a longitudinal axis and a transverse axis, wherein the longitudinal axis of the textile is substantially parallel to the longitudinal axis of the annular frame; and wherein the textile has a proximal end and a distal end and is mounted circumferentially around a first portion of the annular frame, wherein the first portion has a proximal end and a distal end, wherein the proximal end of the first portion is at the inflow end of the annular frame;
wherein at least a portion of the textile comprises at least one composite material comprising a first material and a second material coupled together such that a crimped profile of the implantable medical device is reduced when compared to a substantially identical reference implantable medical device in the absence of the at least one composite material;
wherein the first material exhibits a flexibility and stretchability lower than a flexibility and stretchability of the second material;
wherein the first material has a higher mechanical strength than a mechanical strength of the second material;
and wherein the implantable medical device is configured to radially expand to an expanded configuration from the crimped profile.

2. The implantable medical device of claim 1, wherein the proximal end of the textile is coupled to the proximal end of the first portion of the annular frame, and the distal end of the textile is coupled to the distal end of the first portion of the annular frame.

3. The implantable medical device of claim 1, wherein the first material and the second material are at least partially superposed.

4. The implantable medical device of claim 1, wherein the first material is a woven material and/or wherein the second material is a knitted material.

5. The implantable medical device of claim 1, wherein the second material is at least partially superposed on the first material.

6. The implantable medical device of claim 1,
wherein the first material has a first length and a first width, wherein the first length is measured between a proximal end and a distal end of the first material;
wherein the second material has a second length and a second width, wherein the second length is measured between a proximal end and a distal end of the first material;
wherein the second length is the same or different from the first length; and
wherein the second width is the same or different from the first width.

7. The implantable medical device of claim 6, wherein
the first width is substantially identical to a circumference of the annular frame and:
a) wherein the second width is substantially identical to a circumference of the annular frame, or
b) wherein the second width is smaller than a circumference of the annular frame.

8. The implantable medical device of claim 6, wherein the first length is substantially identical to a length of the first portion of the annular frame, or wherein the first length is shorter than a length of the first portion of the annular frame, or wherein the first length is longer than a length of the first portion of the annular frame.

9. The implantable medical device of claim 6, wherein the second length is substantially identical to a length of the first portion of the annular frame, or wherein the second length is shorter than a length of the first portion of the annular frame, or wherein the second length is longer than a length of the first portion of the annular frame.

10. The implantable medical device of claim 6, wherein the first material and the second material are coupled by an ultrasonic welding such that at least one joining region is formed.

11. The implantable medical device of claim 10 two or more joining regions are disposed randomly or wherein two or more joining regions are disposed in a predetermined pattern.

12. The implantable medical device of claim 10, wherein a thickness of the textile at the at least one of joining regions is different from a thickness of the textile outside of the joining regions.

13. The implantable medical device of claim 11, wherein the two or more joining regions are arranged as a plurality of islands along a length of the composite material and along a circumference of the annular frame.

14. The implantable medical device of claim 11, wherein the two or more joining regions are arranged such that the crimped profile of the implantable medical device is reduced when compared to a substantially identical reference implantable medical device in the absence of the at least one composite material.

15. The implantable medical device of claim 10, wherein the at least one joining region is along the proximal end of the first portion of the annular frame and/or wherein the at least one joining region is along the distal end of the first portion of the annular frame.

16. The implantable medical device of claim 15, wherein the textile is coupled to the proximal end of the first portion of the annular frame through the joining region of the composite material and/or wherein the textile is coupled to the distal end of the first portion of the annular frame through the joining region of the composite material.

17. The implantable medical device of claim 1, wherein the textile further comprises at least a portion where the first and the second materials do not overlay each other.

18. The implantable medical device of claim 1, wherein the surface of the annular frame has a second portion that is free of the textile, and wherein the second portion extends between the outflow end of the annular frame and the distal end of the first portion of the annular frame.

19. The implantable medical device of claim 1, wherein the textile is disposed on an outer surface of the annular frame and is an outer skirt.

20. A method of forming an implantable medical device comprising:
a) providing an annular frame having an inflow end, an outflow end, and a longitudinal axis and comprising a plurality of struts;
b) circumferentially mounting a textile defined by a first surface and an opposite second surface, having a longitudinal axis and a transverse axis, wherein the longitudinal axis of the textile is substantially parallel to the longitudinal axis of the annular frame; and wherein the textile has a proximal end and a distal end and is mounted circumferentially around a first portion of the annular frame, wherein the first portion has a proximal end and a distal end, wherein the proximal end of the first portion is at the inflow end of the annular frame;
wherein at least a portion of the textile comprises at least one composite material comprising a first material and a second material coupled together such that a crimped profile of the implantable medical device is reduced when compared to a substantially identical reference implantable medical device in the absence of the at least one composite material;
wherein the first material exhibits a flexibility and stretchability lower than a flexibility and stretchability of the second material; and
wherein the first material has higher a mechanical strength than a mechanical strength of the second material,
and wherein the implantable medical device is configured to radially expand to an expanded configuration from the crimped profile.
